(19) United States
(12) Patent Application Publication
(10) Pub. No.: US 2024/0116891 A1
(43) Pub. Date: Apr. 11, 2024

(54) MARALIXIBAT SOLID FORMS, INTERMEDIATES AND PROCESSES OF MAKING SAME

(71) Applicant: MIRUM PHARMACEUTICALS, INC., Foster City, CA (US)

(72) Inventor: Pasit PHIASIVONGSA, Hillsborough, CA (US)

(73) Assignee: MIRUM PHARMACEUTICALS, INC., Foster City, CA (US)

(21) Appl. No.: 18/368,041

(22) Filed: Sep. 14, 2023

Related U.S. Application Data
(60) Provisional application No. 63/406,556, filed on Sep. 14, 2022.

(51) Int. Cl. C07D 337/04

(52) U.S. Cl. CPC C07D 337/04; C07B 2200/13

(57) ABSTRACT

The present invention relates to highly pure and crystalline compounds as starting materials and intermediates to make maralixibat and the processes of making highly pure maralixibat. The invention also relates to an amorphous form of maralixibat.

### CROSS REFERENCE TO RELATED APPLICATIONS

This application claims benefit to Provisional Application No. 63/406,556, filed Sep. 14, 2022, which is hereby incorporated by reference in its entirety, and to which application we claim priority.

### FIELD OF THE INVENTION

The present invention relates to highly pure and crystalline compounds as starting materials and intermediates to make maralixibat and the process of making highly pure maralixibat.

### BACKGROUND

Hypercholemia and cholestatic liver diseases are liver diseases associated with impaired bile secretion (i.e., cholestasis), associated with and often secondary to the intracellular accumulation of bile acids/salts in the hepatocyte. Hypercholemia is characterized by increased serum concentration of bile acid or bile salt. Cholestasis can be categorized clinicopathologically into two principal categories of obstructive, often extrahepatic, cholestasis, and nonobstructive, or intrahepatic, cholestasis. Nonobstructive intrahepatic cholestasis can further be classified into two principal subgroups of primary intrahepatic cholestasis that result from constitutively defective bile secretion, and secondary intrahepatic cholestasis that result from hepatocellular injury. Primary intrahepatic cholestasis includes diseases such as benign recurrent intrahepatic cholestasis, which is predominantly an adult form with similar clinical symptoms, and progressive familial intrahepatic cholestasis (PFIC) types 1, 2, and 3, which are diseases that affect children.

Alagille syndrome is an inherited condition in which bile builds up in the liver. One of the major features of Alagille syndrome is liver damage caused by abnormalities in the bile ducts. Alagille syndrome is associated with abnormalities of the liver, heart, skeleton, eye, and kidneys and a characteristic facial appearance.

Maralixibat (as maralixibat chloride) is currently the only approved medication to treat pruritus in people with Alagille syndrome. It is known that maralixibat chloride inhibits apical sodium co-dependent bile acid transport (U.S. Pat. No. 5,994,391). The synthesis of maralixibat chloride is previously disclosed in U.S. patent application Pub. No. 2003/0199515A1. Conventional methods of synthesizing maralixibat chloride resulted in the presence of undesirable structurally related impurities and other solid forms.

Thus, there is a need for a novel process of making highly pure maralixibat from highly pure and crystalline compounds there are starting materials or intermediates in order to minimize formations of impurities. Embodiments of the present disclosure are directed to this and other considerations.

### SUMMARY OF THE INVENTION

Various non-limiting aspects and embodiments of the invention are described below.

Provided herein is a crystalline form of (4R,5R)-3,3-dibutyl-7-(dimethylamino)-4-hydroxy-5-(4-methoxyphenyl)-2,3,4,5-tetrahydrobenzo[b]thiepine 1,1-dioxide (Formula I), which is Form A.

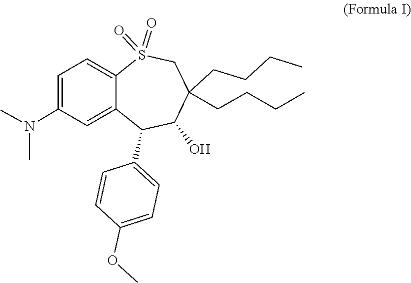 [SMILES: CCCCC1(CCCC)CS(=O)(=O)c2ccc(N(C)C)cc2[C@@H](c2ccc(OC)cc2)[C@H]1O]

Also provided herein is a crystalline form of Formula I, which is Form B.

Also provided herein is a highly pure compound of Formula I, wherein the purity of the compound of Formula I is at least about 95%.

Also provided herein is a crystalline form of (4R,5R)-3,3-dibutyl-7-(dimethylamino)-4-hydroxy-5-(4-hydroxyphenyl)-2,3,4,5-tetrahydrobenzo[b]thiepine 1,1-dioxide (Formula II), which is Form X.

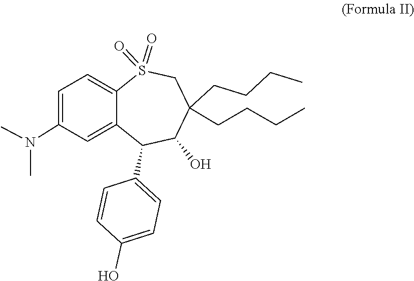 [SMILES: CCCCC1(CCCC)CS(=O)(=O)c2ccc(N(C)C)cc2[C@@H](c2ccc(O)cc2)[C@H]1O]

Also provided herein is a highly pure compound of Formula II, wherein the purity of the compound of Formula II is at least about 95%.

Also provided herein is an amorphous form of 1-(4-((4-((4-R,5R)-3,3-dibutyl-7-(dimethylamino)-4-hydroxy-1,1-dioxido-2,3,4,5-tetrahydrobenzo[b]thiepin-5-yl)phenoxy)methyObenzyl)-1,4-diazabicyclo[2.2.2]octan-1-ium chloride (Formula III)

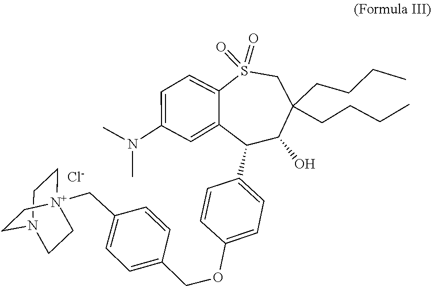 [SMILES: CCCCC1(CCCC)CS(=O)(=O)c2ccc(N(C)C)cc2[C@@H](c2ccc(OCc3ccc(C[N+]45CCN(CC4)CC5)cc3)cc2)[C@H]1O ; [Cl-]]

characterized by at least about 80% amorphous content, wherein the amorphous form is characterized by an XRPD pattern substantially similar to that in FIG. 9.

Also provided herein is a process of making a highly pure compound of Formula III, said process comprising the step of converting a highly pure compound of Formula I to a highly pure compound of Formula II.

Also provided herein is a highly pure crystalline Form I, Form II, and/or amorphous form of Formula III, wherein the form of Formula III is prepared by a process comprising the step of converting a highly pure crystalline Form A of Formula I to a highly pure crystalline Form X of Formula II.

These and other aspects of the present invention will become apparent to those skilled in the art after a reading of the following detailed description of the invention, including the appended claims.

### BRIEF DESCRIPTION OF THE DRAWINGS

Reference will now be made to the accompanying drawings, which are not necessarily drawn to scale, and which illustrate various implementations, aspects, and principles of the disclosed technology.

FIG. 1 is a XRPD plot of Form A of compound of Formula I.

FIG. 2 is a TGA/DSC plot of Form A of compound of Formula I.

FIG. 3 is a PLM image of Form A of compound of Formula I.

FIG. 4 is a XRPD plot of Form B of compound of Formula I.

FIG. 5 is a TGA plot of Form B of compound of Formula I.

FIG. 6 is a XRPD plot of Form X of compound of Formula II.

FIG. 7 is a TGA/DSC plot of Form X of compound of Formula II.

FIG. 8 is a PLM image of Form X of compound of Formula II.

FIG. 9 is a XRPD plot of amorphous form of compound of Formula III.

FIG. 10 is a DSC plot of amorphous form of compound of Formula III.

FIG. 11 is a XRPD plot of Form II of compound of Formula III in comparison to the reference.

FIG. 12 is a TGA/DSC plot of Form II of compound of Formula III.

FIG. 13 is a PLM image of Form II of compound of Formula III.

FIG. 14 is a XRPD plot of Form I of compound of Formula III.

FIG. 15 is a TGA/DSC plot of Form I of compound of Formula III.

### DETAILED DESCRIPTION

Detailed embodiments of the present invention are disclosed herein; however, it is to be understood that the disclosed embodiments are merely illustrative of the invention that may be embodied in various forms. In addition, each of the examples given in connection with the various embodiments of the invention is intended to be illustrative, and not restrictive. Therefore, specific structural and functional details disclosed herein are not to be interpreted as limiting, but merely as a representative basis for teaching one skilled in the art to variously employ the present invention.

Unless defined otherwise, all technical and scientific terms used herein have the same meaning as commonly understood by one of ordinary skill in the art to which this invention belongs.

In some embodiments, if aspects of the disclosure are described as “comprising”, or versions thereof (e.g., comprises), a feature, embodiments also are contemplated “consisting of” or “consisting essentially of” the feature.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

A “subject” or “patient” or “individual” or “animal”, as used herein, refers to humans, veterinary animals (e.g., cats, dogs, cows, horses, sheep, pigs, etc.) and experimental animal models of diseases (e.g., mice, rats). In a preferred embodiment, the subject is a human.

As used herein the term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions of the invention, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

Ranges can be expressed herein as from “about” or “approximately” one particular value and/or to “about” or “approximately” another particular value. When such a range is expressed, another embodiment includes from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

The term “alkyl,” as used herein, alone or in combination, refers to a straight-chain or branched-chain alkyl radical. Alkyl groups may be optionally substituted as defined herein. Examples of alkyl radicals include methyl, ethyl, n-propyl, isopropyl, n-butyl, isobutyl, sec-butyl, tent-butyl, pentyl, iso-amyl, hexyl, octyl, nonyl and the like.

The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “amorphous” refers to a solid form of a molecule and/or ion that is not crystalline. An amorphous solid does not display a definitive X-ray diffraction pattern with sharp maxima.

The term “anhydrous” or “anhydrate” when referring to a crystalline form means that no solvent molecules, including those of water, form a portion of the unit cell of the crystalline form. A sample of an anhydrous crystalline form may nonetheless contain solvent molecules that do not form part of the unit cell of the anhydrous crystalline form, e.g., as residual solvent molecule left behind from the production of the crystalline form. In a preferred embodiment, a solvent can make up 0.5% by weight of the total composition of a sample of an anhydrous form. In a more preferred embodiment, a solvent can make up 0.2% by weight of the total composition of a sample of an anhydrous form. In some embodiments, a sample of an anhydrous crystalline form contains no solvent molecules, e.g., no detectable amount of solvent. The term “solvate” when referring to a crystalline form means that solvent molecules, e.g., organic solvents and water, form a portion of the unit cell of the crystalline form. Solvates that contain water as the solvent are also referred to herein as “hydrates.” The term “isomorphic” when referring to a crystalline form means that the form can comprise different chemical constituents, e.g., contain different solvent molecules in the unit cell, but have identical XRPD patterns. Isomorphic crystalline forms are sometimes referred to herein as “isomorphs.”

As used herein, “crystalline” refers to a solid having a highly regular chemical structure, i.e., having long range structural order in the crystal lattice. The molecules are arranged in a regular, periodic manner in the 3-dimensional space of the lattice. In particular, a crystalline form may be produced as one or more single crystalline forms. A crystalline form of a compound refers to a substantially crystalline form that has at least a particular weight percent crystalline. Particular weight percentages are 70%, 75%, 80%, 85%, 87%, 88%, 89%, 90%, 91%, 92%, 93%, 94%, 95%, 96%, 97%, 98%, 99%, 99.5%, 99.9%, or any percentage between 70% and 100%. In certain embodiments, the particular weight percent of crystallinity is at least 90%. In certain other embodiments, the particular weight percent of crystallinity is at least 95%. In some embodiments, compound of Formula I is a substantially crystalline sample of any of the crystalline solid forms described herein (e.g., Forms A and B). In some embodiments, compound of Formula II is a substantially crystalline sample of the crystalline solid forms described herein (e.g., Forms X and Y). In some embodiments, compound of Formula III is a substantially crystalline sample of the crystalline solid forms described herein (e.g., Forms I and II).

For the purposes of this application, the terms “crystalline form”, “single crystalline form,” “crystalline solid form,” “solid form,” and “polymorph” are synonymous and used interchangeably; the terms distinguish between crystals that have different properties (e.g., different XRPD patterns and/or different DSC scan results).

“Form A” and “Form B”, as used herein, refer to the specific crystal solid state forms of (4R,5R)-3,3-dibutyl-7-(dimethylamino)-4-hydroxy-5-(4-methoxyphenyl)-2,3,4,5-tetrahydrobenzo[b]thiepine 1,1-dioxide (Formula I), wherein the molecules are arranged to form a distinguishable crystal lattice (i) comprising distinguishable unit cells and (ii) yielding distinguishable diffraction peaks when subjected to X-ray radiation.

“Form X” and “Form Y”, as used herein, refer to the specific crystal solid state forms of (4R,5R)-3,3-dibutyl-7-(dimethylamino)-4-hydroxy-5-(4-hydroxyphenyl)-2,3,4,5-tetrahydrobenzo[b]thiepine 1,1-dioxide (Formula II), wherein the molecules are arranged to form a distinguishable crystal lattice (i) comprising distinguishable unit cells and (ii) yielding distinguishable diffraction peaks when subjected to X-ray radiation.

“Form I” and “Form II”, as used herein, refer to the specific crystal solid state forms of 1-(4-((4-((4R,5R)-3,3-dibutyl-7-(dimethylamino)-4-hydroxy-1,1-dioxido-2,3,4,5-tetrahydrobenzo[b]thiepin-5-yl)phenoxy)methyl)benzyl)-1,4-diazabicyclo[2.2.2] octan-1-ium chloride (Formula III), wherein the molecules are arranged to form a distinguishable crystal lattice (i) comprising distinguishable unit cells and (ii) yielding distinguishable diffraction peaks when subjected to X-ray radiation.

The term “highly pure” means the composition of the compound that contains at least about 90% based on the weight of such crystalline form. The term “at least about 90%,” while not intending to limit the applicability of the doctrine of equivalents to the scope of the claims, includes, but is not limited to, for example, about 90, about 91, about 92, about 93, about 94, about 95, about 96, about 97, about 98, about 99 and about 100% wt. %, based on the weight of the crystalline form referred to. The remainder of the composition may comprise other Form(s) of the compound and/or reaction impurities and/or processing impurities that arise, for example, when the crystalline form is prepared. The presence of reaction impurities and/or processing impurities may be determined by analytical techniques known in the art, such as, for example, chromatography, nuclear magnetic resonance spectroscopy, mass spectroscopy, and/or infrared spectroscopy.

“Maralixibat chloride” refers to 1-(4-((4-((4R,5R)-3,3-dibutyl-7-(dimethylamino)-4-hydroxy-1,1-dioxido-2,3,4,5-tetrahydrobenzo[b]thiepin-5-yl)phenoxy)methyl)benzyl)-1,4-diazabicyclo[2.2.2]octan-1-ium chloride, the structure of which is represented in Formula III.

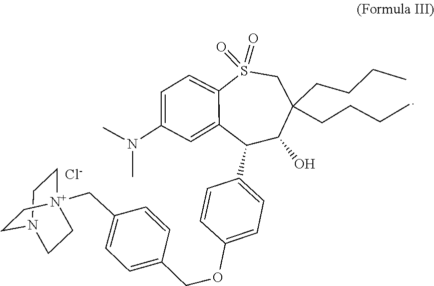 [SMILES: CCCCC1(CCCC)CS(=O)(=O)c2ccc(N(C)C)cc2[C@@H](c2ccc(OCc3ccc(C[N+]45CCN(CC4)CC5)cc3)cc2)[C@H]1O ; [Cl-]]

“Maralixibat” refers to 1-(4-((4-((4R,5R)-3,3-dibutyl-7-(dimethylamino)-4-hydroxy-1,1-dioxido-2,3,4,5-tetrahydrobenzo[b]thiepin-5-yl)phenoxy)methyl)benzyl)-1,4-diazabicyclo[2.2.2]octan-1-ium, which is the free form of maralixibat chloride. The structure of maralixibat is represented below.

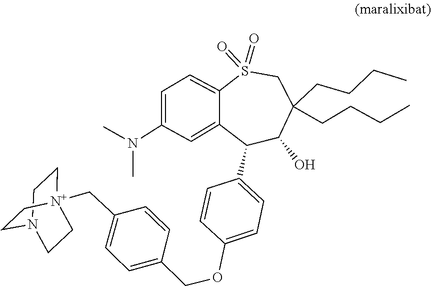 [SMILES: CCCCC1(CCCC)CS(=O)(=O)c2ccc(N(C)C)cc2[C@@H](c2ccc(OCc3ccc(C[N+]45CCN(CC4)CC5)cc3)cc2)[C@H]1O]

The percentage (%), if not otherwise indicated, refers to the weight percentage.

The compounds of the disclosure, or their pharmaceutically acceptable salts can contain one or more asymmetric centers and can thus give rise to enantiomers, diastereomers, and other stereoisomeric forms that can be defined, in terms of absolute stereochemistry, as (R)- or (S)-. The present disclosure is meant to include all such possible isomers, as well as their racemic and optically pure forms whether or not they are specifically depicted herein. Optically active (+) and (−), or (R)- and (S)-isomers can be prepared using chiral synthons or chiral reagents, or resolved using conventional techniques, for example, chromatography and fractional crystallization.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposable mirror images of one another. In one embodiment, compounds disclosed herein include racemic mixtures, enantiomers, diastereomers, or enantiomerically or diasteriomerically enriched mixtures.

“XRPD” refers to X-ray powder diffraction or X-ray powder diffractogram. As used herein, “VT-XRPD” refers to variable temperature-X-ray powder diffraction or variable temperature-X-ray powder diffractogram. As used herein, “TGA” refers to thermogravimetric analysis. As used herein, “DSC” refers to differential scanning calorimetry. As used herein, “NMR” refers to nuclear magnetic resonance. As used herein, “DVS” refers to dynamic vapor sorption. As used herein, “DCM” refers to dichloromethane. As used herein, “EtOAc” refers to ethyl acetate. As used herein, “MeOH” refers to methanol. As used herein, “MBTE” refers to methyl tert-butyl ether. As used herein, “RH” refers to relative humidity. As used herein, “RT” refers to room temperature.

Provided herein is a crystalline form of (4R,5R)-3,3-dibutyl-7-(dimethylamino)-4-hydroxy-5-(4-methoxyphenyl)-2,3,4,5-tetrahydrobenzo[b]thiepine 1,1-dioxide (Formula I)

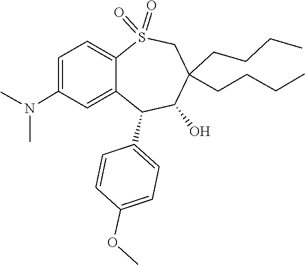 [SMILES: CCCCC1(CCCC)CS(=O)(=O)c2ccc(N(C)C)cc2[C@@H](c2ccc(OC)cc2)[C@H]1O]

which is Form A.

In some embodiments, the crystalline Form A of compound of Formula I is characterized by an X-ray powder diffractogram (XRPD) having a signal of at least three peaks in 2θ values chosen from 4.7±0.2, 6.6±0.2, 9.3±0.2, 10.4±0.2, 13.3±0.2, 16.8±0.2, 19.3±0.2, and 23.4±0.2.

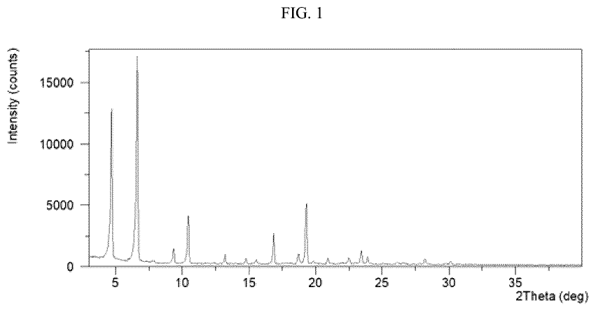

In some embodiments, the crystalline Form A of compound of Formula I is characterized by an XRPD pattern substantially similar to that in FIG. 1.

In some embodiments, the crystalline Form A of compound of Formula I is characterized by a melting point at about 133° C. to about 135° C.

In some embodiments, the crystalline Form A of compound of Formula I is characterized by a melting point at about 134.7° C.

In some embodiments, the crystalline Form A of compound of Formula I is characterized by a DSC thermogram with an endotherm having an onset at about 130.8° C., a peak at about 134.7° C., and an exotherm at about 145° C.

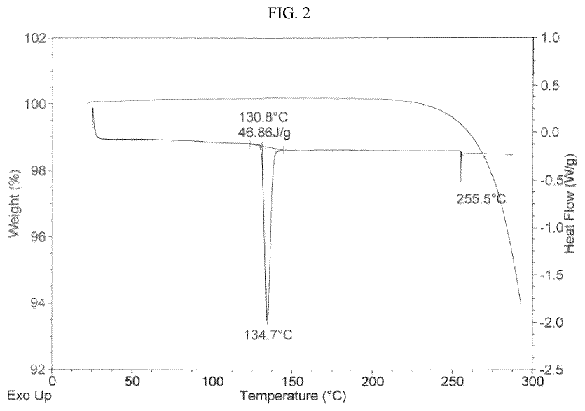

In some embodiments, the crystalline Form A of compound of Formula I is characterized by a thermogravimetric analysis/differential scanning calorimetry (TGA/DSC) thermogram substantially similar to that in FIG. 2.

In some embodiments, the crystalline Form A of compound of Formula I has no weight loss between about 25° C. to about 225° C. as measured by thermogravimetric analysis (TGA).

In some embodiments, the crystalline Form A of compound of Formula I is anhydrous.

In some embodiments, the crystalline Form A of compound of Formula I is a hydrate.

In some embodiments, the crystalline Form A of compound of Formula I comprises rod-shaped crystals as observed by polarized light microscopy.

In some embodiments, at least about 95% of the crystalline Form A of compound of Formula I is the R,R stereoisomer.

In some embodiments, at least about 96% of the crystalline Form A of compound of Formula I is the R,R stereoisomer.

In some embodiments, at least about 97% of the crystalline Form A of compound of Formula I is the R,R stereoisomer.

In some embodiments, at least about 98% of the crystalline Form A of compound of Formula I is the R,R stereoisomer.

In some embodiments, at least about 99% of the crystalline Form A of compound of Formula I is the R,R stereoisomer.

In some embodiments, about 100% of the crystalline Form A of compound of Formula I is the R,R stereoisomer.

In some embodiments, about 99% to about 100% of the crystalline Form A of compound of Formula I is the R,R stereoisomer.

Also provided herein is a crystalline form of (4R,5R)-3,3-dibutyl-7-(dimethylamino)-4-hydroxy-5-(4-methoxyphenyl)-2,3,4,5-tetrahydrobenzo[b]thiepine 1,1-dioxide (Formula I).

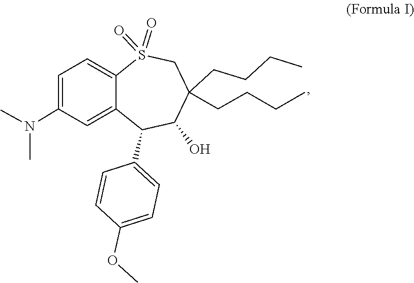 [SMILES: CCCCC1(CCCC)CS(=O)(=O)c2ccc(N(C)C)cc2[C@@H](c2ccc(OC)cc2)[C@H]1O]

which is Form B.

In some embodiments, the crystalline Form B of compound of Formula I is characterized by an X-ray powder diffractogram (XRPD) having a signal of at least five peaks in 2θ values chosen from 4.4±0.2, 6.3±0.2, 7.2±0.2, 8.9±0.2, 9.9±0.2, 10.8 +0.2, 11.2 +0.2, 11.6±0.2, 17.5±0.2, 18.4±0.2, 21.9±0.2 and at least two peaks in 2θ values chosen from 4.7±0.2, 6.6±0.2, 9.3±0.2, 13.3±0.2, 19.3±0.2, and 23.4±0.2.

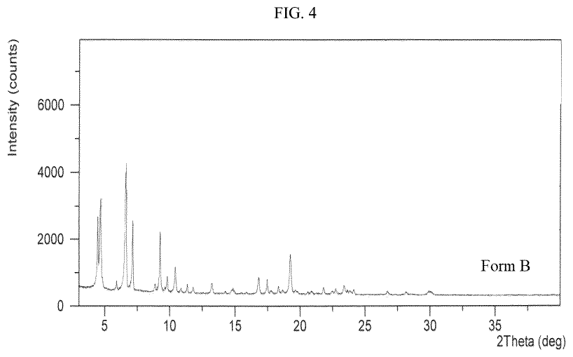

In some embodiments, the crystalline Form B of compound of Formula I is characterized by an XRPD pattern substantially similar to that in FIG. 4.

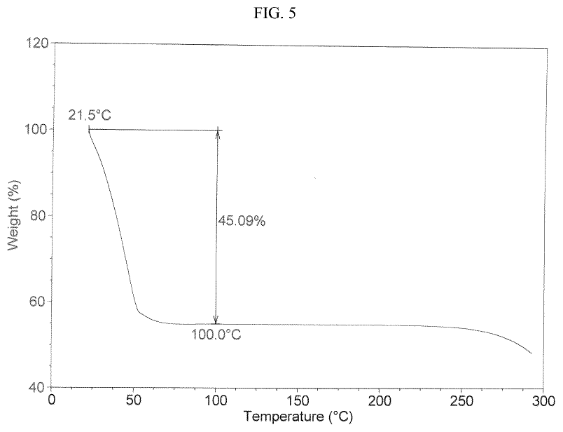

In some embodiments, the crystalline Form B of compound of Formula I is characterized by a TGA thermogram substantially similar to that in FIG. 5.

In some embodiments, the crystalline Form B of compound of Formula I has about 45% weight loss between about 21.5° C. to about 100° C. as measured by thermogravimetric analysis (TGA).

In some embodiments, the crystalline Form B of compound of Formula I is a hydrate.

In some embodiments, at least about 95% of the crystalline Form B of compound of Formula I is the R,R stereoisomer.

In some embodiments, at least about 96% of the crystalline Form B of compound of Formula I is the R,R stereoisomer.

In some embodiments, at least about 97% of the crystalline Form B of compound of Formula I is the R,R stereoisomer.

In some embodiments, at least about 98% of the crystalline Form B of compound of Formula I is the R,R stereoisomer.

In some embodiments, at least about 99% of the crystalline Form B of compound of Formula I is the R,R stereoisomer.

In some embodiments, about 100% of the crystalline Form B of compound of Formula I is the R,R stereoisomer.

In some embodiments, about 99% to about 100% of the crystalline Form B of compound of Formula I is the R,R stereoisomer.

Also provided herein is a highly pure compound (4R,5R)-3,3-dibutyl-7-(dimethylamino)-4-hydroxy-5-(4-methoxyphenyl)-2,3,4,5-tetrahydrobenzo[b]thiepine 1,1-dioxide having the structure of Formula I,

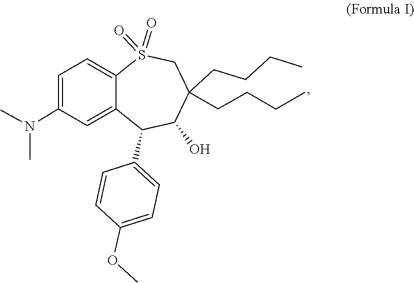 [SMILES: CCCCC1(CCCC)CS(=O)(=O)c2ccc(N(C)C)cc2[C@@H](c2ccc(OC)cc2)[C@H]1O]

wherein the purity of the compound of Formula I is at least about 95%.

In some embodiments, the purity of the compound of Formula I is at least about 96%.

In some embodiments, the purity of the compound of Formula I is at least about 97%.

In some embodiments, the purity of the compound of Formula I is at least about 98%.

In some embodiments, the purity of the compound of Formula I is at least about 99%.

In some embodiments, the purity of the compound of Formula I is about 100%.

In some embodiments, the purity of the compound of Formula I is about 99% to about 100%.

In some embodiments, the highly pure compound of Formula I is in a crystalline form.

In some embodiments, the highly pure compound of Formula I is in an amorphous form.

In some embodiments, the highly pure compound of Formula I is in Form A.

In some embodiments, the highly pure compound of Formula I is in Form B.

In some embodiments, the highly pure compound of Formula I comprise Form A and Form B.

Also provided herein is a process of preparing the crystalline Form A comprising (a) mixing the compound of Formula I with a solvent.

In some embodiments, the process of preparing the crystalline Form A further comprises (b) stirring the mixture in a slurry at about 25° C. In some embodiments, the process of preparing the crystalline Form A further comprises (b) stirring the mixture in a slurry at about 65° C. In some embodiments, step (b) further comprises stirring the mixture in a slurry for about 3 days.

In some embodiments, the process of preparing the crystalline Form A has step (a) that further comprises obtaining a clear solution of the mixture, and the process further comprises (b) adding an antisolvent. In some embodiments, step (b) further comprises stirring the mixture at about 25° C. In some embodiments, the solvent of step (a) is a volatile solvent, and step (a) further comprises contacting the solid form of the compound of Formula I with the vapor of the volatile solvent. In some embodiments, the contacting with the vapor of the volatile solvent is at about 25° C. In some embodiments, the contacting lasts for about 7 days.

In some embodiments, the process of preparing the crystalline Form A has step (a) that further comprises obtaining a clear solution of the mixture and the process further comprises (b) contacting the mixture with the vapor of a volatile antisolvent. In some embodiments, the contacting with the vapor of the volatile antisolvent is at about 25° C. In some embodiments, the contacting lasts for about 7 days.

In some embodiments, the process of preparing the crystalline Form A has step (a) that further comprises obtaining a clear solution of the mixture, and the process further comprises (b) evaporating the solvent. In some embodiments, the evaporation is at about 25° C. In some embodiments, the evaporation lasts for about 7 days.

In some embodiments, the process of preparing the crystalline Form A has step (a) that further comprises obtaining a clear solution of the mixture, and the process further comprises (b) adding an ionic liquid. In some embodiments, the ionic liquid is 1-butyl-3-methyl imidazolium chloride.

In some embodiments, the process of preparing the crystalline Form A further comprises (c) evaporating the solvent. In some embodiments, the evaporation is at about 25° C. In some embodiments, the evaporation lasts for about 7 days.

In some embodiments, the process of preparing the crystalline Form A further comprises (d) placing the mixture at about 5° C. In some embodiments, step (d) comprises placing the mixture at about 5° C. for about 3 days.

In some embodiments, the process of preparing the crystalline Form A comprises step (a) further comprising forming a saturated solution, and the process further comprises (b) cooling the solution to form a clear solution. In some embodiments, the cooling is from about 50° C. to about 5° C. In some embodiments, the cooling time is at range of about 6 hours to about 9 hours.

In some embodiments, the process of preparing the crystalline Form A further comprises (c) evaporating the solvent. In some embodiments, the evaporation is at about 25° C. In some embodiments, the evaporation lasts for about 5 days.

In some embodiments, the process of preparing the crystalline Form A further comprises (d) placing the mixture at about −14° C. In some embodiments, step (d) comprises placing the mixture at about −14° C. for about 3 days.

In some embodiments, the process of preparing the crystalline Form A comprises the solvent of step (a) comprising one or more selected from the group consisting of acetone, acetonitrile (ACN), n-butyl acetate, cyclohexane, dichloromethane (DCM), dimethylformamide (DMF), dimethylsulfoxide (DMSO), ethyl acetate (EtOAc), ethanol (EtOH), water, n-heptane, isopropyl alcohol (IPA), isopropyl acetate (IPAc), methanol (MeOH), methyltetrahydrofuran (MeTHF), methyl isobutyl ketone (MIBK), methyl tert-butyl ether (MTBE), NMP, pentane, and toluene.

In some embodiments, the process of preparing the crystalline Form A comprises the solvent of step (a) selected from the group consisting of cyclohexane, water, n-heptane, IPA, MTBE, pentane, cyclohexane/acetone (3:2, volume:volume), cyclohexane/DCM (3:2, volume:volume), water/DMSO (3:2, volume:volume), water/EtOH (3:2, volume:volume), n-heptane/toluene (3:2 volume:volume), IPA/toluene (3:2, volume:volume), MTBE/DMSO (3:2, volume:volume), pentane/DCM (3:2, volume:volume), and pentane/acetone (3:2, volume:volume)

In some embodiments, the process of preparing the crystalline Form A comprises the solvent of step (a) selected from the group consisting of cyclohexane, water, n-heptane, IPA, MTBE, pentane, cyclohexane/toluene (3:2, volume: volume), cyclohexane/DCM (3:2, volume:volume), n-heptane/butyl acetate (3:2, volume:volume), n-heptane/toluene (3:2, volume:volume), IPA/acetone (2:1, volume:volume), IPA/DMF (2:1, volume:volume), MTBE/ACN (2:1, volume:volume), MTBE/DMSO (2:1, volume:volume), and MTBE/n-butyl acetate (2:1, volume:volume).

In some embodiments, the process of preparing the crystalline Form A comprises the antisolvent of step (b) selected from the group consisting of cyclohexane, water, n-heptane, IPA, MTBE, and pentane.

In some embodiments, the process of preparing the crystalline Form A comprises the volatile solvent selected from the group consisting of acetone, ACN, n-butyl acetate, DCM, DMF, DMSO, EtOAc, MeOH, NMP, and MIBK.

In some embodiments, the process of preparing the crystalline Form A comprises the solvent of step (a) that is n-butyl acetate, EtOAc, or toluene.

In some embodiments, the process of preparing the crystalline Form A comprises the antisolvent of step (b) selected from the group consisting of cyclohexane, water, n-heptane, IPA, MTBE, and pentane.

In some embodiments, the process of preparing the crystalline Form A comprise the solvent of step (a) selected from the group consisting of DMF, MeTHF, MIBK, NMP, toluene, DCM, acetone, ACN, n-butyl acetate, and DMSO.

In some embodiments, the process of preparing the crystalline Form A comprise the solvent of step (a) selected from the group consisting of EtOH, IPAc, MeOH, MTBE, MIBK, CAN, Acetone, NMP, toluene, and MeTHF.

Also provided herein is a process of preparing the crystalline Form B, said process comprising (a) mixing the compound of Formula I with a solvent, and (b) adding an antisolvent, wherein the antisolvent is a mixture of acetonitrile (ACN) and water.

Also provided herein is a crystalline form of (4R,5R)-3,3-dibutyl-7-(dimethylamino)-4-hydroxy-5-(4-hydroxyphenyl)-2,3,4,5-tetrahydrobenzo[b]thiepine 1,1-dioxide (Formula II)

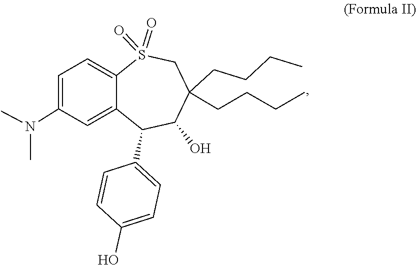 [SMILES: CCCCC1(CCCC)CS(=O)(=O)c2ccc(N(C)C)cc2[C@@H](c2ccc(O)cc2)[C@H]1O]

which is Form X.

In some embodiments, the crystalline Form X of compound of Formula II is characterized by an X-ray powder diffractogram (XRPD) having a signal of at least nine peaks in 2θ values chosen from 5.0±0.2, 8.7±0.2, 10.0±0.2, 12.5±0.2, 13.3±0.2, 14.9±0.2, 15.8±0.2, 17.0±0.2, 17.8±0.2, 19.3±0.2, 19.9±0.2, 21.7±0.2, 22.6±0.2, 23.2±0.2, 24.5±0.2, 24.9±0.2, 25.8±0.2, 26.4±0.2, 28.0±0.2, and 29.4±0.2.

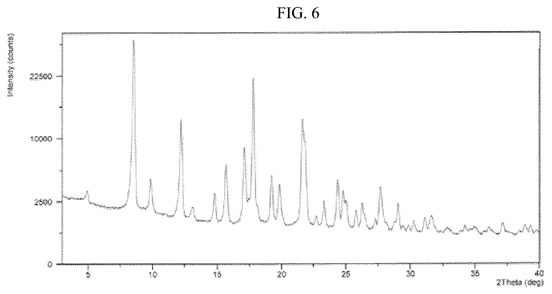

In some embodiments, the crystalline Form X of compound of Formula II is characterized by an XRPD pattern substantially similar to that in FIG. 6.

In some embodiments, the crystalline Form X of compound of Formula II is characterized by a melting point at about 216.7° C. to about 217.7° C.

In some embodiments, the crystalline Form X of compound of Formula II is characterized by a melting point at about 217.2° C.

In some embodiments, the crystalline Form X of compound of Formula II is characterized by a DSC thermogram with an endotherm having an onset at about 216.1° C., a peak at about 217.2° C., and an exotherm at about 218.3° C.

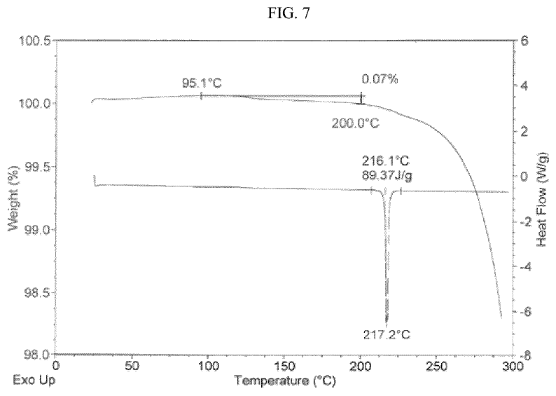

In some embodiments, the crystalline Form X of compound of Formula II is characterized by a TGA/DSC thermogram substantially similar to that in FIG. 7.

In some embodiments, the crystalline Form X of compound of Formula II has less than about 0.1% weight loss between about 95.1° C. to about 200.0° C. as measured by thermogravimetric analysis (TGA).

In some embodiments, the crystalline Form X of compound of Formula II has about 0.07% weight loss between about 95.1° C. to about 200.0° C. as measured by thermogravimetric analysis (TGA).

In some embodiments, the crystalline Form X of compound of Formula II is anhydrous.

In some embodiments, the crystalline Form X of compound of Formula II is a hydrate.

In some embodiments, the crystalline Form X of compound of Formula II comprises rod-shaped crystals as observed by polarized light microscopy.

In some embodiments, at least about 95% of the crystalline Form X of compound of Formula II is the R,R stereoisomer.

In some embodiments, at least about 96% of the crystalline Form X of compound of Formula II is the R,R stereoisomer.

In some embodiments, at least about 97% of the crystalline Form X of compound of Formula II is the R,R stereoisomer.

In some embodiments, at least about 98% of the crystalline Form X of compound of Formula II is the R,R stereoisomer.

In some embodiments, at least about 99% of the crystalline Form X of compound of Formula II is the R,R stereoisomer.

In some embodiments, about 100% of the crystalline Form X of compound of Formula II is the R,R stereoisomer.

In some embodiments, about 99% to about 100% the crystalline Form X of compound of Formula II is the R,R stereoisomer.

Also provided herein is a highly pure compound (4R,5R)-3,3-dibutyl-7-(dimethylamino)-4-hydroxy-5-(4-hydroxyphenyl)-2,3,4,5-tetrahydrobenzo[b]thiepine 1,1-dioxide (Formula II)

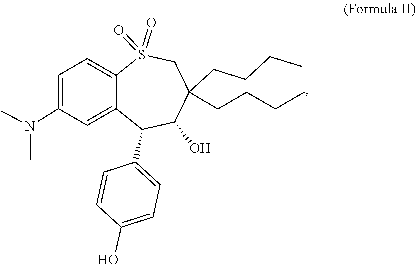 [SMILES: CCCCC1(CCCC)CS(=O)(=O)c2ccc(N(C)C)cc2[C@@H](c2ccc(O)cc2)[C@H]1O]

wherein the purity of the compound of Formula II is at least about 95%.

In some embodiments, the purity of the compound of Formula II is at least about 96%.

In some embodiments, the purity of the compound of Formula II is at least about 97%.

In some embodiments, the purity of the compound of Formula II is at least about 98%.

In some embodiments, the purity of the compound of Formula II is at least about 99%.

In some embodiments, the purity of the compound of Formula II is about 100%.

In some embodiments, the purity of the compound of Formula II is about 99% to about 100%.

In some embodiments, the highly pure compound of Formula II is in a crystalline form.

In some embodiments, the highly pure compound of Formula II is in an amorphous form.

In some embodiments, the highly pure compound of Formula II is in Form X.

In some embodiments, the highly pure compound of Formula II is in Form Y.

In some embodiments, the highly pure compound of Formula II comprise Form X and Form Y.

Also provided herein is a process of preparing the highly pure compound of Formula II, wherein said process comprises contacting a highly pure compound (4R,5R)-3,3-dibutyl-7-(dimethylamino)-4-hydroxy-5-(4-methoxyphenyl)-2,3,4,5-tetrahydrobenzo[b]thiepine 1,1-dioxide (Formula I) with DL-methionine to form a mixture, wherein the highly pure compound of Formula I has a purity of at least 95%.

In some embodiments, the process of preparing the highly pure compound of Formula II further comprises contacting the mixture with CH₃SO₃H at about 85° C.

In some embodiments, the process of preparing the highly pure compound of Formula II comprises contacting the compound of Formula I at the purity of at least about 96%.

In some embodiments, the process of preparing the highly pure compound of Formula II comprises contacting the compound of Formula I at the purity of at least about 97%.

In some embodiments, the process of preparing the highly pure compound of Formula II comprises contacting the compound of Formula I at the purity of at least about 98%.

In some embodiments, the process of preparing the highly pure compound of Formula II comprises contacting the compound of Formula I at the purity of at least about 99%.

In some embodiments, the process of preparing the highly pure compound of Formula II comprises contacting the compound of Formula I at the purity of about 100%.

In some embodiments, the process of preparing the highly pure compound of Formula II comprises contacting the compound of Formula I at the purity of about 99% to about 100%.

In some embodiments, the process prepares the highly pure compound of Formula II comprises contacting the compound of Formula I in a crystalline form. In some embodiments, the crystalline form of the compound of Formula I is Form A. In some embodiments, the crystalline form of the compound of Formula I is Form B.

Also provided herein is a process of preparing the crystalline Form X comprising (a) mixing the compound of Formula II with a solvent.

In some embodiments, the process of preparing the crystalline Form X further comprises (b) stirring the mixture in a slurry at about 25° C. In some embodiments, the process of preparing the crystalline Form X further comprises (b) stirring the mixture in a slurry at about 65° C. In some embodiments, step (b) further comprises stirring the mixture in a slurry for about 3 days.

In some embodiments, the process of preparing the crystalline Form X comprises step (a) that further comprises obtaining a clear solution of the mixture, and the process further comprises (b) adding an antisolvent. In some embodiments, step (b) further comprises stirring the mixture at about 25° C. In some embodiments, the solvent of step (a) is a volatile solvent, and step (a) further comprises contacting the solid form of the compound of Formula II with the vapor of the volatile solvent. In some embodiments, the contacting with the vapor of the volatile solvent is at about 25° C. In some embodiments, the contacting lasts for about 7 days.

In some embodiments, the process of preparing the crystalline Form X comprises step (a) that further comprises obtaining a clear solution of the mixture and the process further comprises (b) contacting the mixture with the vapor of a volatile antisolvent. In some embodiments, the contacting with the vapor of the volatile antisolvent is at about 25° C. In some embodiments, the contacting lasts for about 7 days.

In some embodiments, the process of preparing the crystalline Form X comprises step (a) that further comprises obtaining a clear solution of the mixture, and the process further comprises (b) evaporating the solvent. In some embodiments, the evaporation is at about 25° C. In some embodiments, the evaporation lasts for about 7 days.

In some embodiments, the process of preparing the crystalline Form X comprises step (a) that further comprises obtaining a clear solution of the mixture, and the process further comprises (b) adding an ionic liquid. In some embodiments, the ionic liquid is 1-butyl-3-methyl imidazolium chloride.

In some embodiments, the process of preparing the crystalline Form X further comprises (c) evaporating the solvent. In some embodiments, the evaporation is at about 25° C. In some embodiments, the evaporation lasts for about 7 days.

In some embodiments, the process of preparing the crystalline Form X further comprises (d) placing the mixture at about 5° C. In some embodiments, step (d) comprises placing the mixture at about 5° C. for about 3 days.

In some embodiments, the process of preparing the crystalline Form X comprises step (a) further comprising forming a saturated solution, and the process further comprises (b) cooling the solution to form a clear solution. In some embodiments, the cooling is from about 50° C. to about 5° C. In some embodiments, the cooling time is at range of about 6 hours to about 9 hours.

In some embodiments, the process of preparing the crystalline Form X further comprises (c) evaporating the solvent. In some embodiments, the evaporation is at about 25° C. In some embodiments, the evaporation lasts for about 5 days.

In some embodiments, the process of preparing the crystalline Form X further comprises (d) placing the mixture at about −14° C. In some embodiments, step (d) comprises placing the mixture at about −14° C. for about 3 days.

In some embodiments, the process of preparing the crystalline Form X comprises the solvent of step (a) comprising one or more selected from the group consisting of acetone, acetonitrile (ACN), n-butyl acetate, cyclohexane, dichloromethane (DCM), dimethylformamide (DMF), dimethylsulfoxide (DMSO), ethyl acetate (EtOAc), ethanol (EtOH), water, n-heptane, isopropyl alcohol (IPA), isopropyl acetate (IPAc), methanol (MeOH), methyltetrahydrofuran (MeTHF), methyl isobutyl ketone (MIBK), methyl tert-butyl ether (MTBE), NMP, pentane, and toluene.

In some embodiments, the process of preparing the crystalline Form X comprises the solvent of step (a) selected from the group consisting of cyclohexane, water, n-heptane, IPA, MTBE, EtOH, pentane, toluene, cyclohexane/acetone (3:2, volume:volume), water/DMF (2:1, volume:volume), n-heptane/ethyl acetate (3:2, volume:volume), pentane/acetone (3:2, volume:volume), toluene/DMSO (3:2, volume:volume), toluene/DMF (3:2, volume:volume), and butyl acetate/ACN (1:1, volume:volume).

In some embodiments, the process of preparing the crystalline Form X comprises the solvent of step (a) selected from the group consisting of cyclohexane, water, n-heptane, IPA, MTBE, EtOH, pentane, toluene, Butyl acetate/DCM (1:1, volume:volume), cyclohexane/butyl acetate (1:1, volume:volume), EtOH/DMF (3:2, volume:volume), EtOH/DMSO (3:2, volume:volume), n-heptane/acetone (3:2, volume:volume), water/DMF (3:2, volume:volume), and water/DMSO (3:3 volume:volume).

In some embodiments, the process of preparing the crystalline Form X comprises the antisolvent of step (b) selected from the group consisting of cyclohexane, water, n-heptane, IPA, MTBE, and pentane.

In some embodiments, the process of preparing the crystalline Form X comprises the antisolvent of step (b) selected from the group consisting of water, n-heptane, toluene, and pentane.

In some embodiments, the process of preparing the crystalline Form X comprises the volatile solvent selected from the group consisting of acetone, DMF, DMSO, EtOAc, MeTHF, NMP, MIBK, DMF/ethyl acetate (3:2, volume:volume), DMSO/ethyl acetate (3:2, volume:volume), and acetone/DMF (2:3 volume:volume).

In some embodiments, the process of preparing the crystalline Form X comprises the solvent of step (a) is DMF, DMSO, acetone, and EtOAc.

In some embodiments, the process of preparing the crystalline Form X comprises the antisolvent of step (b) selected from the group consisting of toluene, water, n-heptane, and pentane.

In some embodiments, the process of preparing the crystalline Form X comprises the solvent of step (a) selected from the group consisting of NMP, acetone, DMF, DMSO, EtOAc, MeTHF, MIBK, DMF/ethyl acetate (3:2, volume:volume), and DMSO/ethyl acetate (3:2, volume:volume).

In some embodiments, the process of preparing the crystalline Form X comprises the solvent of step (a) selected from the group consisting of EtOH, IPAc, MeOH, MTBE, MIBK, CAN, Acetone, NMP, toluene, and MeTHF.

In some embodiments, the process of preparing the crystalline Form X comprises the solvent of step (a) selected from the group consisting of NMP, acetone, DMF, DMSO, EtOAc, MeTHF, MIBK, acetone/DMF (2:3, volume:volume), acetone/DMSO (2:3, volume:volume), and DMF/ethyl acetate (3:2, volume:volume).

In some embodiments, the process of preparing the crystalline Form X comprises the solvent of step (a) selected from the group consisting of DMSO, EtOAc, MeTHF, MIBK, NMP, DMF, DMF/EtOAc (3:2, volume:volume), DMSO/EtOAc (3:2, volume:volume), acetone/DMF (2:3, volume:volume), and acetone/DMSO (2:3 volume:volume).

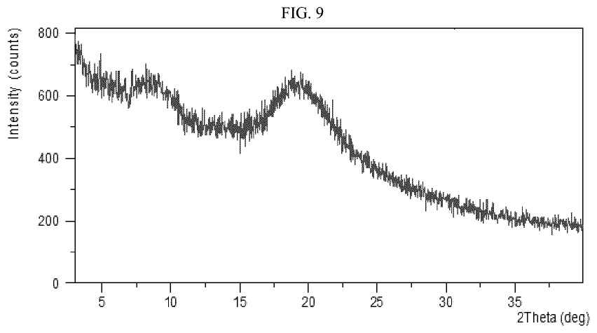

Also provided herein is an amorphous form of 1-(4-((4-((4R,5R)-3,3-dibutyl-7-(dimethylamino)-4-hydroxy-1,1-dioxido-2,3,4,5-tetrahydrobenzo[b]thiepin-5-yl)phenoxy)methyl)benzyl)-1,4-diazabicyclo[2.2.2]octan-1-ium chloride (Formula III) characterized by at least about 80% amorphous content. In some embodiments, the amorphous form of compound of Formula III is characterized by an XRPD pattern substantially similar to that in FIG. 9.

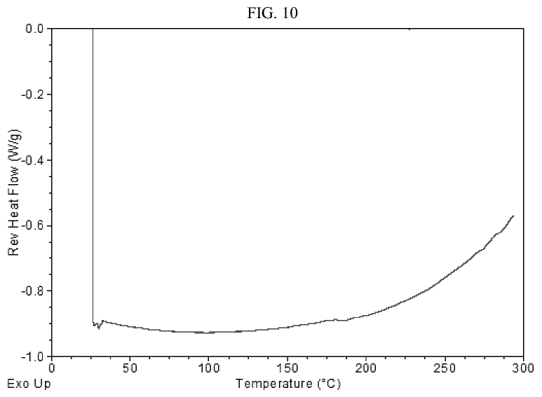

In some embodiments, the amorphous form of compound of Formula III is characterized by a DSC thermogram substantially similar to that in FIG. 10.

In some embodiments, the amorphous form of compound of Formula III is characterized by at least about 85% amorphous content.

In some embodiments, the amorphous form of compound of Formula III is characterized by at least about 90% amorphous content.

In some embodiments, the amorphous form of compound of Formula III is characterized by at least about 95% amorphous content.

In some embodiments, the amorphous form of compound of Formula III is characterized by at least about 96% amorphous content.

In some embodiments, the amorphous form of compound of Formula III is characterized by at least about 97% amorphous content.

In some embodiments, the amorphous form of compound of Formula III is characterized by at least about 98% amorphous content.

In some embodiments, the amorphous form of compound of Formula III is characterized by at least about 99% amorphous content.

In some embodiments, the amorphous form of compound of Formula III is characterized by about 100% amorphous content.

Also provided herein is a process of making the amorphous form, said process comprising (a) mixing the compound of Formula III with methanol, and (b) evaporating the solvent by means of rotary evaporation.

Also provided herein is a process of making a highly pure compound 1-(4-((4-((4R,5R)-3,3-dibutyl-7-(dimethylamino)-4-hydroxy-1,1-dioxido-2,3,4,5-tetrahydrobenzo[b]thiepin-5-yl)phenoxy)methyl)benzyl)-1,4-diazabicyclo[2.2.2]octan-1-ium chloride (Formula III)

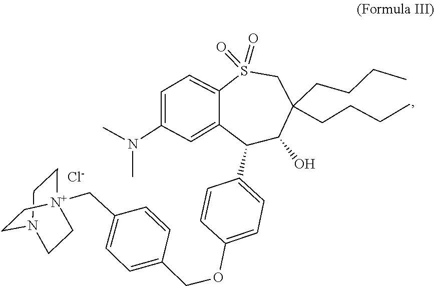 [SMILES: CCCCC1(CCCC)CS(=O)(=O)c2ccc(N(C)C)cc2[C@@H](c2ccc(OCc3ccc(C[N+]45CCN(CC4)CC5)cc3)cc2)[C@H]1O ; [Cl-]]

said process comprising the step of converting a highly pure compound (4R,5R)-3,3-dibutyl-7-(dimethylamino)-4-hydroxy-5-(4-methoxyphenyl)-2,3,4,5-tetrahydrobenzo[b]thiepine 1,1-dioxide (Formula I)

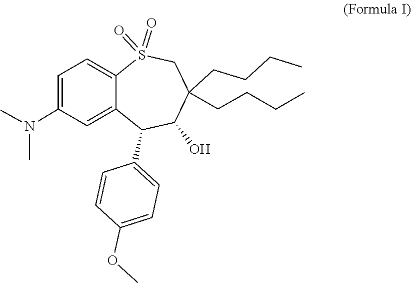 [SMILES: CCCCC1(CCCC)CS(=O)(=O)c2ccc(N(C)C)cc2[C@@H](c2ccc(OC)cc2)[C@H]1O]

to a highly pure compound (4R,5R)-3,3-dibutyl-7-(dimethylamino)-4-hydroxy-5-(4-hydroxyphenyl)-2,3,4,5-tetrahydrobenzo[b]thiepine 1,1-dioxide (Formula II)

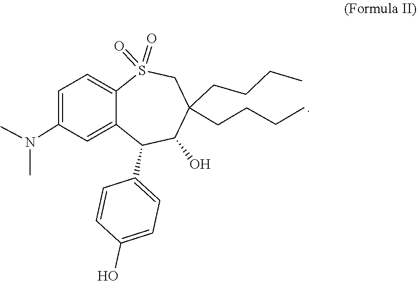 [SMILES: CCCCC1(CCCC)CS(=O)(=O)c2ccc(N(C)C)cc2[C@@H](c2ccc(O)cc2)[C@H]1O]

In some embodiments, the process of making the highly pure compound of Formula III comprises the step of converting a highly pure compound of Formula I of a purity of at least about 90%.

In some embodiments, the process of making the highly pure compound of Formula III comprises the step of converting a highly pure compound of Formula I of a purity of at least about 95%.

In some embodiments, the process of making the highly pure compound of Formula III comprises the step of converting a highly pure compound of Formula I of a purity of at least about 96%.

In some embodiments, the process of making the highly pure compound of Formula III comprises the step of converting a highly pure compound of Formula I of a purity of at least about 97%.

In some embodiments, the process of making the highly pure compound of Formula III comprises the step of converting a highly pure compound of Formula I of a purity of at least about 98%.

In some embodiments, the process of making the highly pure compound of Formula III comprises the step of converting a highly pure compound of Formula I of a purity of at least about 98.5%.

In some embodiments, the process of making the highly pure compound of Formula III comprises the step of converting a highly pure compound of Formula I of a purity of at least about 99%.

In some embodiments, the process of making the highly pure compound of Formula III comprises the step of converting a highly pure compound of Formula I of a purity of about 100%.

In some embodiments, the process of making the highly pure compound of Formula III comprises the step of converting a highly pure compound of Formula I to a highly pure compound of Formula II of a purity of at least about 90%.

In some embodiments, the process of making the highly pure compound of Formula III comprises the step of converting a highly pure compound of Formula I to a highly pure compound of Formula II of a purity of at least about 95%.

In some embodiments, the process of making the highly pure compound of Formula III comprises the step of converting a highly pure compound of Formula I to a highly pure compound of Formula II of a purity of at least about 96%.

In some embodiments, the process of making the highly pure compound of Formula III comprises the step of converting a highly pure compound of Formula I to a highly pure compound of Formula II of a purity of at least about 97%.

In some embodiments, the process of making the highly pure compound of Formula III comprises the step of converting a highly pure compound of Formula I to a highly pure compound of Formula II of a purity of at least about 98%.

In some embodiments, the process of making the highly pure compound of Formula III comprises the step of converting a highly pure compound of Formula I to a highly pure compound of Formula II of a purity of at least about 98.5%.

In some embodiments, the process of making the highly pure compound of Formula III comprises the step of converting a highly pure compound of Formula I to a highly pure compound of Formula II of a purity of at least about 99%.

In some embodiments, the process of making the highly pure compound of Formula III comprises the step of converting a highly pure compound of Formula I to a highly pure compound of Formula II of a purity of about 100%.

In some embodiments, the process of making the highly pure compound of Formula III of a purity of at least about 90%.

In some embodiments, the process of making the highly pure compound of Formula III of a purity of at least about 95%.

In some embodiments, the process of making the highly pure compound of Formula III of a purity of at least about 96%.

In some embodiments, the process of making the highly pure compound of Formula III of a purity of at least about 97%.

In some embodiments, the process of making the highly pure compound of Formula III of a purity of at least about 98%.

In some embodiments, the process of making the highly pure compound of Formula III of a purity of at least about 98.5%.

In some embodiments, the process of making the highly pure compound of Formula III of a purity of at least about 99%.

In some embodiments, the process of making the highly pure compound of Formula III of a purity of about 100%.

In some embodiments, the process of making the highly pure compound of Formula III comprises the step of converting a highly pure compound of Formula I, wherein the compound of Formula I is in crystalline Form.

In some embodiments, the process of making the highly pure compound of Formula III comprises the step of converting a highly pure compound of Formula I, wherein the compound of Formula I is in crystalline Form A.

In some embodiments, the process of making the highly pure compound of Formula III comprises the step of converting a highly pure compound of Formula I to a highly pure compound of Formula II, wherein the compound of Formula II is in crystalline Form.

In some embodiments, the process of making the highly pure compound of Formula III comprises the step of converting a highly pure compound of Formula I to a highly pure compound of Formula II, wherein the compound of Formula II is in crystalline Form X.

In some embodiments, the process of making the highly pure compound of Formula III comprises the step of converting a highly pure compound of Formula I to a highly pure compound of Formula II, wherein the compound of Formula III is in crystalline Form.

In some embodiments, the process of making the highly pure compound of Formula III comprises the step of converting a highly pure compound of Formula I to a highly pure compound of Formula II, wherein the compound of Formula III is in crystalline Form I.

In some embodiments, the process of making the highly pure compound of Formula III comprises the step of converting a highly pure compound of Formula I to a highly pure compound of Formula II, wherein the compound of Formula III is in crystalline Form II.

Provided herein is a process of making a highly pure compound 1-(4-((4-((4R,5R)-3,3-dibutyl-7-(dimethylamino)-4-hydroxy-1,1-dioxido-2,3,4,5-tetrahydrobenzo[b]thiepin-5-yl)phenoxy)methyObenzyl)-1,4-diazabicyclo[2.2.2]octan-1-ium chloride (Formula III)

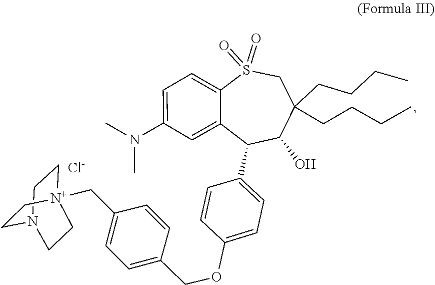 [SMILES: CCCCC1(CCCC)CS(=O)(=O)c2ccc(N(C)C)cc2[C@@H](c2ccc(OCc3ccc(C[N+]45CCN(CC4)CC5)cc3)cc2)[C@H]1O ; [Cl-]]

said process comprising the step of converting the crystalline Form A of compound (4R,5R)-3,3-dibutyl-7-(dimethylamino)-4-hydroxy-5-(4-methoxyphenyl)-2,3,4,5-tetrahydrobenzo[b]thiepine 1,1-dioxide (Formula I)

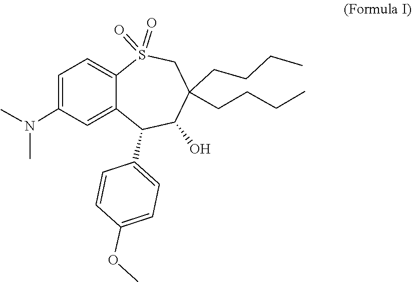 [SMILES: CCCCC1(CCCC)CS(=O)(=O)c2ccc(N(C)C)cc2[C@@H](c2ccc(OC)cc2)[C@H]1O]

to the crystalline Form X of compound (4R,5R)-3,3-dibutyl-7-(dimethylamino)-4-hydroxy-5-(4-hydroxyphenyl)-2,3,4,5-tetrahydrobenzo[b]thiepine 1,1-dioxide (Formula II).

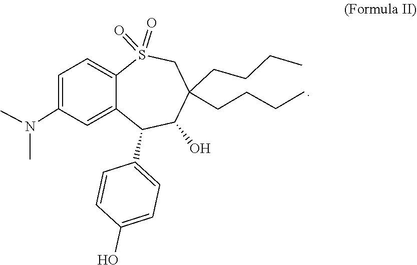 [SMILES: CCCCC1(CCCC)CS(=O)(=O)c2ccc(N(C)C)cc2[C@@H](c2ccc(O)cc2)[C@H]1O]

In some embodiments, the process of making the highly pure compound of Formula III of a purity of at least about 90%.

In some embodiments, the process of making the highly pure compound of Formula III of a purity of at least about 95%.

In some embodiments, the process of making the highly pure compound of Formula III of a purity of at least about 96%.

In some embodiments, the process of making the highly pure compound of Formula III of a purity of at least about 97%.

In some embodiments, the process of making the highly pure compound of Formula III of a purity of at least about 98%.

In some embodiments, the process of making the highly pure compound of Formula III of a purity of at least about 98.5%.

In some embodiments, the process of making the highly pure compound of Formula III of a purity of at least about 99%.

In some embodiments, the process of making the highly pure compound of Formula III of a purity of about 100%.

In some embodiments, the process of making the highly pure compound of Formula III in crystalline Form. In some embodiments, In some embodiments, the process of making the highly pure compound of Formula III in crystalline Form I. In some embodiments, the process of making the highly pure compound of Formula III in crystalline Form II.

### Methods of Use

Also provided herein is a method of treating cholestatic liver disease or condition in a subject in need thereof, comprising administering a therapeutically effective amount of the highly pure crystalline Form II of the compound of Formula III made by the process described above.

Also provided herein is a method of treating cholestatic liver disease or condition in a subject in need thereof, comprising administering a therapeutically effective amount of the highly pure crystalline Form II of the compound of Formula III made by the process described above.

Also provided herein is a method of treating cholestatic liver disease or condition in a subject in need thereof, comprising administering a therapeutically effective amount of the amorphous form of compound of Formula III.

In some embodiments, the cholestatic liver disease is progressive familial intrahepatic cholestasis (PFIC), biliary atresia, Alagille syndrome (ALGS), intrahepatic cholestasis of pregnancy (ICP), or any pediatric cholestatic condition resulting in below normal growth, height, or weight. In some embodiments, the cholestatic liver disease is biliary atresia. In various embodiments, the cholestatic liver disease is PFIC. In various embodiments, the PFIC is selected from PFIC type 1, PFIC type 2, PFIC type 3, PFIC type 4, PFIC type 5, and PFIC type 6. In some embodiments, the PFIC is selected from PFIC type 1, PFIC type 2, and PFIC type 3. In some embodiments, the PFIC is PFIC type 2. In some embodiments, the subject has an ABCB11 gene with a missense mutation and no truncating mutation. In some embodiments, the cholestatic liver disease is ALGS. In some embodiments, the cholestatic liver disease is PFIC. In various embodiments, the cholestatic liver disease is ICP.

In some embodiments, the cholestatic liver disease or condition for treatment is selected from the group consisting of obstructive cholestasis, non-obstructive cholestasis, extrahepatic cholestasis, intrahepatic cholestasis, primary intrahepatic cholestasis, secondary intrahepatic cholestasis, progressive familial intrahepatic cholestasis (PFIC), PFIC type 1, PFIC type 2, PFIC type 3, benign recurrent intrahepatic cholestasis (BRIC), BRIC type 1, BRIC type 2, BRIC type 3, total parenteral nutrition associated cholestasis, paraneoplastic cholestasis, Stauffer syndrome, intrahepatic cholestasis of pregnancy, contraceptive-associated cholestasis, drug-associated cholestasis, infection-associated cholestasis, Dubin-Johnson Syndrome, primary biliary cirrhosis (PBC), primary sclerosing cholangitis (PSC), gallstone disease, Alagille syndrome, Dubin-Johnson Syndrome, biliary atresia, post-Kasai biliary atresia, post-liver transplantation biliary atresia, post-liver transplantation cholestasis, post-liver transplantation associated liver disease, intestinal failure associated liver disease, bile acid mediated liver injury, MRP2 deficiency syndrome, and neonatal sclerosing cholangitis.

In some embodiments, the cholestatic liver disease or condition is Alagille syndrome (ALGS).

### Alagille Syndrome

Alagille syndrome is a genetic disorder that affects the liver and other organs. It often presents during infancy (e.g., age 6-18 months) through early childhood (e.g., age 3-5 years) and may stabilize after the age of 10. Symptoms may include chronic progressive cholestasis, ductopenia, jaundice, pruritus, xanthomas, congenital heart problems, paucity of intrahepatic bile ducts, poor linear growth, hormone resistance, posterior embryotoxon, Axenfeld anomaly, retinitis pigmentosa, pupillary abnormalities, cardiac murmur, atrial septal defect, ventricular septal defect, patent ductus arteriosus, and Tetralogy of Fallot. Individuals diagnosed with Alagille syndrome have been treated with ursodiol, hydroxyzine, cholestyramine, rifampicin, and phenobarbitol. Due to a reduced ability to absorb fat-soluble vitamins, individuals with Alagille Syndrome are further administered high dose multivitamins.

In some embodiments, the cholestatic liver disease is associated with progressive familial intrahepatic cholestasis (PFIC).

### Progressive Familial Intrahepatic Cholestasis (PFIC)

PFIC is a rare genetic disorder that causes progressive liver disease typically leading to liver failure. In people with PFIC, liver cells are less able to secrete bile. The resulting buildup of bile causes liver disease in affected individuals. Signs and symptoms of PFIC typically begin in infancy Patients experience severe itching, jaundice, failure to grow at the expected rate (failure to thrive), and an increasing inability of the liver to function (liver failure). The disease is estimated to affect one in every 50,000 to 100,000 births in the United States and Europe. Six types of PFIC have been genetically identified, all of which are similarly characterized by impaired bile flow and progressive liver disease.

### PFIC 1

PFIC 1 (also known as, Byler disease or FIC1 deficiency) is associated with mutations in the ATP8B1 gene (also designated as FIC1). This gene, which encodes a P-type ATPase, is located on human chromosome 18 and is also mutated in the milder phenotype, benign recurrent intrahepatic cholestasis type 1 (BRIO) and in Greenland familial cholestasis. FIC1 protein is located on the canalicular membrane of the hepatocyte but within the liver it is mainly expressed in cholangiocytes. P-type ATPase appears to be an aminophospholipid transporter responsible for maintaining the enrichment of phosphatidylserine and phophatidylethanolamme on the inner leaflet of the plasma membrane in comparison of the outer leaflet. The asymmetric distribution of lipids in the membrane bilayer plays a protective role against high bile salt concentrations in the canalicular lumen. The abnormal protein function may indirectly disturb the biliary secretion of bile acids. The anomalous secretion of bile acids/salts leads to hepatocyte bile acid overload.

PFIC 1 typically presents in infants (e.g., age 6-18 months). The infants may show signs of pruritus, jaundice, abdominal distension, diarrhea, malnutrition, and shortened stature. Biochemically, individuals with PFIC 1 have elevated serum transaminases, elevated bilirubin, elevated serum bile acid levels, and low levels of gammaGT. The individual may also have liver fibrosis. Individuals with PFIC 1 typically do not have bile duct proliferation. Most individuals with PFIC 1 will develop end-stage liver disease by 10 years of age. No medical treatments have proven beneficial for the long-term treatment of PFIC 1. In order to reduce extrahepatic symptoms (e.g., malnutrition and failure to thrive), children are often administered medium chain triglycerides and fat-soluble vitamins. Ursodiol has not been demonstrated as effective in individuals with PFIC 1.

### PFIC 2

PFIC 2 (also known as, Byler Syndrome, BSEP deficiency) is associated with mutations in the ABCB11 gene (also designated BSEP). The ABCB11 gene encodes the ATP-dependent canalicular bile salt export pump (BSEP) of human liver and is located on human chromosome 2. BSEP protein, expressed at the hepatocyte canalicular membrane, is the major exporter of primary bile acids/salts against extreme concentration gradients. Mutations in this protein are responsible for the decreased biliary bile salt secretion described in affected patients, leading to decreased bile flow and accumulation of bile salts inside the hepatocyte with ongoing severe hepatocellular damage.

PFIC 2 typically presents in infants (e.g., age 6-18 months). The infants may show signs of pruritus. Biochemically, individuals with PFIC 2 have elevated serum transaminases, elevated bilirubin, elevated serum bile acid levels, and low levels of gammaGT. The individual may also have portal inflammation and giant cell hepatitis. Further, individuals often develop hepatocellular carcinoma. No medical treatments have proven beneficial for the long-term treatment of PFIC 2. In order to reduce extrahepatic symptoms (e.g., malnutrition and failure to thrive), children are often administered medium chain triglycerides and fat-soluble vitamins. The PFIC 2 patient population accounts for approximately 60% of the PFIC population.

### PFIC 3

PFIC 3 (also known as MDR3 deficiency) is caused by a genetic defect in the ABCB4 gene (also designated MDR3) located on chromosome 7. Class III Multidrug Resistance (MDR3) P-glycoprotein (P-gp), is a phospholipid translocator involved in biliary phospholipid (phosphatidylcholine) excretion in the canlicular membrane of the hepatocyte. PFIC 3 results from the toxicity of bile in which detergent bile salts are not inactivated by phospholipids, leading to bile canaliculi and biliary epithelium injuries.

PFIC 3 also presents in early childhood. As opposed to PFIC 1 and PFIC 2, individuals have elevated gammaGT levels. Individuals also have portal inflammation, fibrosis, cirrhosis, and massive bile duct proliferation. Individuals may also develop intrahepatic gallstone disease. Ursodiol has been effective in treating or ameliorating PFIC 3.

### PFIC 4

PFIC 4 (also known as beta-hydroxy-delta-5-C27-steroid oxidoreductase deficiency or TJP2 deficiency) is caused by homozygous or compound heterozygous mutation in the TJP2 gene (607709) on chromosome 9q21. The TJP2 protein (Tight Junction Protein 2, sometimes called ZO2) plays a role in “tight junctions”. Tight junctions are areas where the membranes of two adjacent cells join to form a barrier. The barrier controls what molecules are able to pass between cells. Such junctions are important throughout the body, and TJP2 is not specific to the liver. A mild form of liver disease associated with mutations in the TPJ 2 gene was previously called familial hypercholanaemia (which means high bile salts in blood). Only a small number of patients with PFIC caused by TJP2 mutation have been studied so far, so it is not yet understood of what manifestations, other than liver disease and its consequences that TJP2 deficiency patients may have.

Children with TJP2 related cholestasis (PFIC-4) have a variable spectrum of presentation. Some have a self-limiting disease, while others have progressive liver disease with an increased risk of hepatocellular carcinoma. Hence, frequent surveillance for hepatocellular carcinoma is recommended from infancy.

### PFIC 5

PFIC 5 (also known as FXR deficiency) is caused by a mutation in the NR1H4 gene, which encodes the FXR (the Farnesoid X Receptor) protein. This protein is important in regulation of bile acid metabolism in the liver and intestine, as well as in other aspects of metabolism. Patients with PFIC due to FXR deficiency (PFIC 5) seem to develop rapidly progressing liver disease potentially very early in infancy Few patients have been reported so far, although it is expected more will likely be identified as this cause of PFIC is fairly newly described.

PFIC-5 patients usually have rapidly progressive liver disease with early onset coagulopathy, high alpha-fetoprotein and ultimately require a liver transplant.

### PFIC 6

PFIC 6, also known as MYOSB deficiency, is the newest identified PFIC type. MYOSB is involved in maintaining proper functioning of cell membranes and helping to move proteins, such as BSEP, to where they are needed. MY05B has been related to intestinal disorder, cholestasis, or both. Some patients with cholestasis due to MYO5B deficiency have progressive liver disease, while others have it only intermittently. Subjects with MYO5 B-related disease can present with isolated cholestasis or cholestasis with intractable diarrhea (MVID). These children are at risk of worsening cholestasis post intestinal transplant (IT) for MVID, hence combined intestinal and liver transplant or IT with biliary diversion is preferred.

Immunohistochemistry can differentiate most of the variants of PFIC but confirmation requires genetic analysis.

### Benign Recurrent Intrahepatic Cholestasis (BRIC)

### BRIC 1

BRIC1 is caused by a genetic defect of the FIC1 protein in the canalicular membrane of hepatocytes. BRIC1 is typically associated with normal serum cholesterol and γ-glutamyltranspeptidase levels, but elevated serum bile salts. Residual FIC1 expression and function is associated with BRIC1. Despite recurrent attacks of cholestasis or cholestatic liver disease, there is no progression to chronic liver disease in a majority of patients. During the attacks, the patients are severely jaundiced and have pruritis, steatorrhea, and weight loss. Some patients also have renal stones, pancreatitis, and diabetes.

### BRIC 2

BRIC2 is caused by mutations in ABCB11, leading to defective BSEP expression and/or function in the canalicular membrane of hepatocytes.

### BRIC 3

BRIC3 is related to the defective expression and/or function of MDR3 in the canalicular membrane of hepatocytes. Patients with MDR3 deficiency usually display elevated serum γ-glutamyltranspeptidase levels in the presence of normal or slightly elevated bile acid levels.

### Biliary Atresia

Biliary atresia is a life-threatening condition in infants in which the bile ducts inside or outside the liver do not have normal openings. With biliary atresia, bile becomes trapped, builds up, and damages the liver. The damage leads to scarring, loss of liver tissue, and cirrhosis. Without treatment, the liver eventually fails, and the infant needs a liver transplant to stay alive. The two types of biliary atresia are fetal and perinatal. Fetal biliary atresia appears while the baby is in the womb. Perinatal biliary atresia is much more common and does not become evident until 2 to 4 weeks after birth.

### Post-Kasai Biliary Atresia

Biliary atresia is treated with surgery called the Kasai procedure or a liver transplant. The Kasai procedure is usually the first treatment for biliary atresia. During a Kasai procedure, the pediatric surgeon removes the infant's damaged bile ducts and brings up a loop of intestine to replace them. While the Kasai procedure can restore bile flow and correct many problems caused by biliary atresia, the surgery doesn't cure biliary atresia. If the Kasai procedure is not successful, infants usually need a liver transplant within 1 to 2 years. Even after a successful surgery, most infants with biliary atresia slowly develop cirrhosis over the years and require a liver transplant by adulthood. Possible complications after the Kasai procedure include ascites, bacterial cholangitis, portal hypertension, and pruritis.

### Post Liver Transplantation Biliary Atresia

If the atresia is complete, liver transplantation is the only option. Although liver transplantation is generally successful at treating biliary atresia, liver transplantation may have complications such as organ rejection. Also, a donor liver may not become available. Further, in some patients, liver transplantation may not be successful at curing biliary atresia.

### Xanthoma

Xanthoma is a skin condition associated with cholestatic liver diseases, in which certain fats build up under the surface of the skin. Cholestasis results in several disturbances of lipid metabolism resulting in formation of an abnormal lipid particle in the blood called lipoprotein X. Lipoprotein X is formed by regurgitation of bile lipids into the blood from the liver and does not bind to the LDL receptor to deliver cholesterol to cells throughout the body as does normal LDL. Lipoprotein X increases liver cholesterol production by five-fold and blocks normal removal of lipoprotein particles from the blood by the liver.

### Acquired Cholestatic Disease

### Pediatric Primary Sclerosing Cholangitis (PSC)

Pediatric PSC is a chronic inflammatory hepatic disorder slowly progressing to end stage liver failure in most of the affected patients. In pediatric PSC inflammation, fibrosis and obstruction of large and medium sized intra- and extrahepatic ductuli is predominant.

### Gallstone Disease

Gallstone disease is one of the most common and costly of all digestive diseases with a prevalence of up to 17% in Caucasian women. Cholesterol containing gallstones are the major form of gallstones and supersaturation of bile with cholesterol is therefore a prerequisite for gallstone formation. ABCB4 mutations may be involved in the pathogenesis of cholesterol gallstone disease.

### Drug Induced Cholestasis

Inhibition of BSEP function by drugs is an important mechanism of drug-induced cholestasis, leading to the hepatic accumulation of bile salts and subsequent liver cell damage. Several drugs have been implicated in BSEP inhibition. Most of these drugs, such as rifampicin, cyclosporine, glibenclamide, or troglitazone directly cis-inhibit ATP-dependent taurocholate transport in a competitive manner, while estrogen and progesterone metabolites indirectly trans-inhibits BSEP after secretion into the bile canaliculus by Mrp2. Alternatively, drug-mediated stimulation of MRP2 can promote cholestasis or cholestatic liver disease by changing bile composition.

### Total Parenteral Nutrition Associated Cholestasis

TPNAC is one of the most serious clinical scenarios where cholestasis or cholestatic liver disease occurs rapidly and is highly linked with early death. Infants, who are usually premature and who have had gut resections are dependent upon TPN for growth and frequently develop cholestasis or cholestatic liver disease that rapidly progresses to fibrosis, cirrhosis, and portal hypertension, usually before 6 months of life. The degree of cholestasis or cholestatic liver disease and chance of survival in these infants have been linked to the number of septic episodes, likely initiated by recurrent bacterial translocation across their gut mucosa. Although there are also cholestatic effects from the intravenous formulation in these infants, septic mediators likely contribute the most to altered hepatic function.

In some embodiments, the compound of Formula III decreases serum bile acid or hepatic bile acid levels in the patient by at least about 20%, at least about 30%, or at least about 40%.

In some embodiments, less than 10% of the compound of Formula III is systemically absorbed upon oral administration.

In some embodiments, the compound of Formula III is administered at a dosage of between about 10 μg/kg/day and about 10 mg/kg/day.

In some embodiments, the compound of Formula III is administered at a dosage from about 140 μg/kg/day to about 1 mg/kg/day.

In some embodiments, the compound of Formula III is administered at a dosage from about 280 μg/kg/day to about 800 μg/kg/day.

In some embodiments, the compound of Formula III is administered at a dosage comprising from about 0.1 mg to about 40 mg.

In some embodiments, the compound of Formula III decreases the levels of serum bile acids or hepatic bile acids, reduces bilirubin, reduces liver enzymes, lowers intraenterocyte bile acids/salts, or reduces necrosis and/or damage to hepatocellular architecture.

In some embodiments, the compound of Formula III decreases pruritus.

In some embodiments, the compound of Formula III is administered with a second agent selected from a bile acid sequestrant or binder.

In some embodiments, the compound of Formula III is administered before ingestion of food.

In some embodiments, the compound of Formula III is administered less than about 60 minutes or less than about 30 minutes before ingestion of food.

In some embodiments, the compound of Formula III is administered orally.

In some embodiments, the compound of Formula III is administered as an ileal-pH sensitive release or an enterically coated formulation.

In some embodiments, the compound of Formula III is administered with a vitamin supplement.

In some embodiments, the vitamin supplement comprises a fat-soluble vitamin.

In some embodiments, the fat-soluble vitamin is selected from the group consisting of vitamin A, D, E, or K.

Also provided herein is a highly pure crystalline Form I, Form II, and/or amorphous form of 1-(4-((4-((4R,5R)-3,3-dibutyl-7-(dimethylamino)-4-hydroxy-1,1-dioxido-2,3,4,5-tetrahydrobenzo[b]thiepin-5-yl)phenoxy)methyl)benzyl)-1,4-diazabicyclo[2.2.2]octan-1-ium chloride (Formula III), wherein the form of Formula III is prepared by a process comprising the step of converting a highly pure crystalline Form A of (4R,5R)-3,3-dibutyl-7-(dimethylamino)-4-hydroxy-5-(4-methoxyphenyl)-2,3,4,5-tetrahydrobenzo[b] thiepine 1,1-dioxide (Formula I) to a highly pure crystalline Form X of (4R,5R)-3,3-dibutyl-7-(dimethylamino)-4-hydroxy-5-(4-hydroxyphenyl)-2,3,4,5-tetrahydrobenzo[b]thiepine 1,1-dioxide (Formula II).

Also provide herein is a method of treating cholestatic liver disease or condition in a subject in need thereof, comprising administering a therapeutically effective amount of the highly pure crystalline Form I, Form II, and/or amorphous form of the compound of Formula III.

Also provided herein is a composition of 1-(4-((4-((4R,5R)-3,3-dibutyl-7-(dimethylamino)-4-hydroxy-1,1-dioxido-2,3,4,5-tetrahydrobenzo[b]thiepin-5-yl)phenoxy)methyl)benzyl)-1,4-diazabicyclo[2.2.2] octan-1-ium chloride (Formula III),

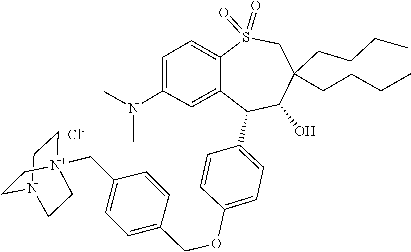 [SMILES: CCCCC1(CCCC)CS(=O)(=O)c2ccc(N(C)C)cc2[C@@H](c2ccc(OCc3ccc(C[N+]45CCN(CC4)CC5)cc3)cc2)[C@H]1O ; [Cl-]]

that comprises one or more of the following: (a) about ≤1.0% to about ≤0.30% or about ≤0.01% of 1-(4-((4-((4S,5S)-3,3-dibutyl-7-(dimethylamino)-4-hydroxy-1,1-dioxido-2,3,4,5-tetrahydrobenzo[b]thiepin-5-yl)phenoxy)methyl)benzyl)-1,4-diazabicyclo[2.2.2]octan-1-ium (Compound VI-Cl-A);

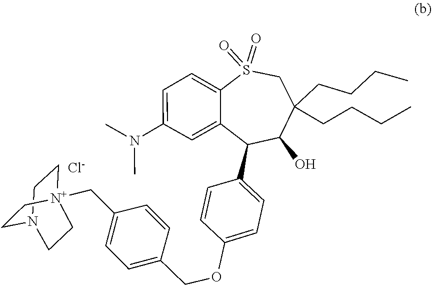 [SMILES: CCCCC1(CCCC)CS(=O)(=O)c2ccc(N(C)C)cc2[C@H](c2ccc(OCc3ccc(C[N+]45CCN(CC4)CC5)cc3)cc2)[C@@H]1O ; [Cl-]]

about ≤0.50% to about ≤0.30% or about ≤0.01% of 1-(4-(((4-((4-((4R,5R)-3,3-dibutyl-7-(dimethylamino)-4-hydroxy-1,1-dioxido-2,3,4,5-tetrahydrobenzo[b]thiepin-5-yl)phenoxy)methyl)benzyl)oxy)methyl)benzyl)-1,4-diazabicyclo[2.2.2] octan-1-ium (Compound VI-Cl-B);

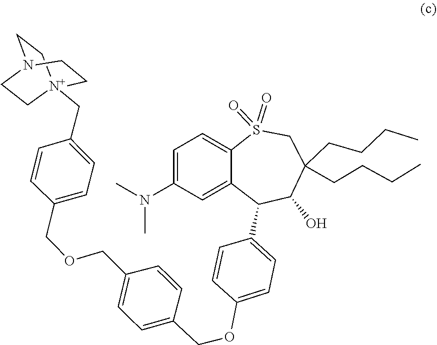 [SMILES: CCCCC1(CCCC)CS(=O)(=O)c2ccc(N(C)C)cc2[C@@H](c2ccc(OCc3ccc(COCc4ccc(C[N+]56CCN(CC5)CC6)cc4)cc3)cc2)[C@H]1O]

about ≤0.50% to about ≤0.30% or about ≤0.01% of 1-(4-((4-((4R,5R)-3,3-dibutyl-4-hydroxy-7-(methylamino)-1,1-dioxido-2,3,4,5-tetrahydrobenzo[b]thiepin-5-yl)phenoxy)methyl)benzyl)-1,4-diazabicyclo[2.2.2]octan-1-ium (Compound VI-Cl-C);

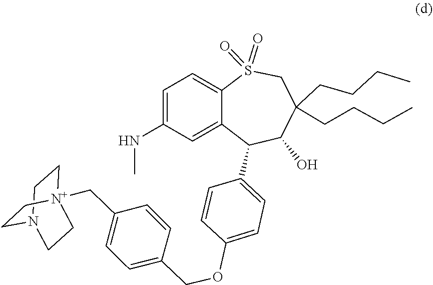 [SMILES: CCCCC1(CCCC)CS(=O)(=O)c2ccc(NC)cc2[C@@H](c2ccc(OCc3ccc(C[N+]45CCN(CC4)CC5)cc3)cc2)[C@H]1O]

about ≤0.50% to about ≤0.30% or about ≤0.01% of 1-(4-((4-((4R,5R)-3,3-dibutyl-4-hydroxy-7-(N-methylformamido)-1,1-dioxido-2,3,4,5-tetrahydrobenzo[b] thiepin-5-yl)phenoxy)methyl)benzyl)-1,4-diazabicyclo[2.2.2]octan-1-ium (Compound VI-Cl-D);

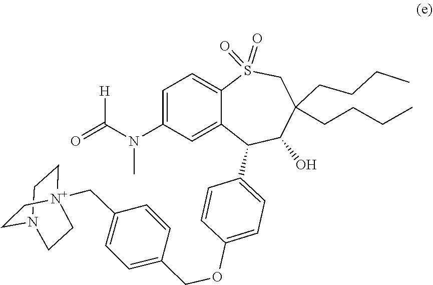 [SMILES: CCCCC1(CCCC)CS(=O)(=O)c2ccc(N(C)C=O)cc2[C@@H](c2ccc(OCc3ccc(C[N+]45CCN(CC4)CC5)cc3)cc2)[C@H]1O]

about ≤0.50% to about ≤0.30% or about ≤0.01% of (4R,5R)-3,3-dibutyl-7-(dimethylamino)-4-hydroxy-5-(4-hydroxyphenyl)-2,3,4,5-tetrahydrobenzo[b]thiepine 1,1-dioxide (Formula II);

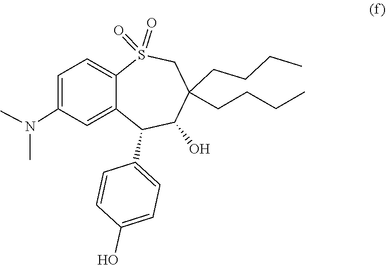 [SMILES: CCCCC1(CCCC)CS(=O)(=O)c2ccc(N(C)C)cc2[C@@H](c2ccc(O)cc2)[C@H]1O]

about ≤0.50% to about ≤0.30% or about ≤0.01% of (4R,5R)-3,3-dibutyl-7-(dimethylamino)-4-hydroxy-5-(4-((4-(hydroxymethyl)benzyl)oxy)phenyl)-2,3,4,5-tetrahydrobenzo[b]thiepine 1,1-dioxide (Formula VIII);

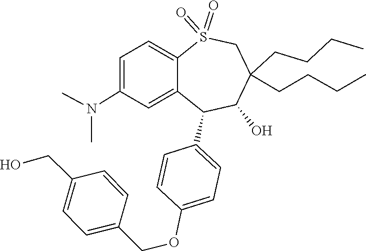 [SMILES: CCCCC1(CCCC)CS(=O)(=O)c2ccc(N(C)C)cc2[C@@H](c2ccc(OCc3ccc(CO)cc3)cc2)[C@H]1O]

and (g) about ≤0.50% to about ≤0.30% of 1,1′-(1,4-phenylenebis(methylene))bis(1,4-diazabicyclo[2.2.2]octan-1-ium)

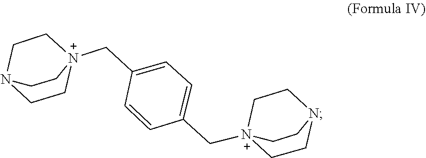 [SMILES: c1cc(C[N+]23CCN(CC2)CC3)ccc1C[N+]12CCN(CC1)CC2]

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

### EXAMPLES

### Example 1. Instruments and Methods

### 1.1 XRPD

XRPD was performed with a Panalytical X'Pert Powder XRPD on a Si zero-background holder. The 20 position was calibrated against a Panalytical Si reference standard disc. Instrumental parameters used are listed in Table 1-1.

TABLE 1-1
Parameters used for XRPD analysis

| | |
|---|---|
| P | Reflection Mode |
| X-Ray waveleng | Cu, kα |
| | Kα1 (Å): 1.540598, |
| | Kα2 (Å): 1.544426, |
| | Kα2/Kαl intensity ratio: 0.50 |
| X-Ray tube se | 45 kV, 40 mA |
| Divergence | Fixed 1/8° |
| Scan m | Continuous |
| Scan range (2θ) | 3-40° |
| Scan step time (s) | 18.87 |
| Step size (2θ) | 0.0131° |
| Test Time | 4 min 15 s |

### 1.2 TGA/DSC

TGA data was collected using a TA Discovery 550 TGA from TA Instrument. DSC was performed using a TA Q2000 DSC from TA Instrument. DSC was calibrated with Indium reference standard and the TGA was calibrated using nickel reference standard, Detailed parameters used are listed in Table 1-2.

TABLE 1-2
Parameters used for TGA and DSC analysis

| Parameters | TGA | DSC |
|---|---|---|
| Method | Ramp | Ramp |
| Sample pan | Platinum, open | Aluminum, crimped |

| | |
|---|---|
| Temperature | RT - 300° C. |
| Heating rate | 10° C./min |
| Purge gas | N₂ |

### 1.3 PL

Polarized light microscopic (PLM) picture was captured on a Nikon DS-Fi2 upright microscope at room temperature. Low viscosity microscope immersion oil (Resolve®) was used to disperse powder crystals. The following examples use the instruments and methods described in Example 1.

### Example 2. Synthesis and Characterization of Compound of Formula I

### 2.1 Synthesis of Compound of Formula I

The synthetic process is outlined in the flow diagram below.

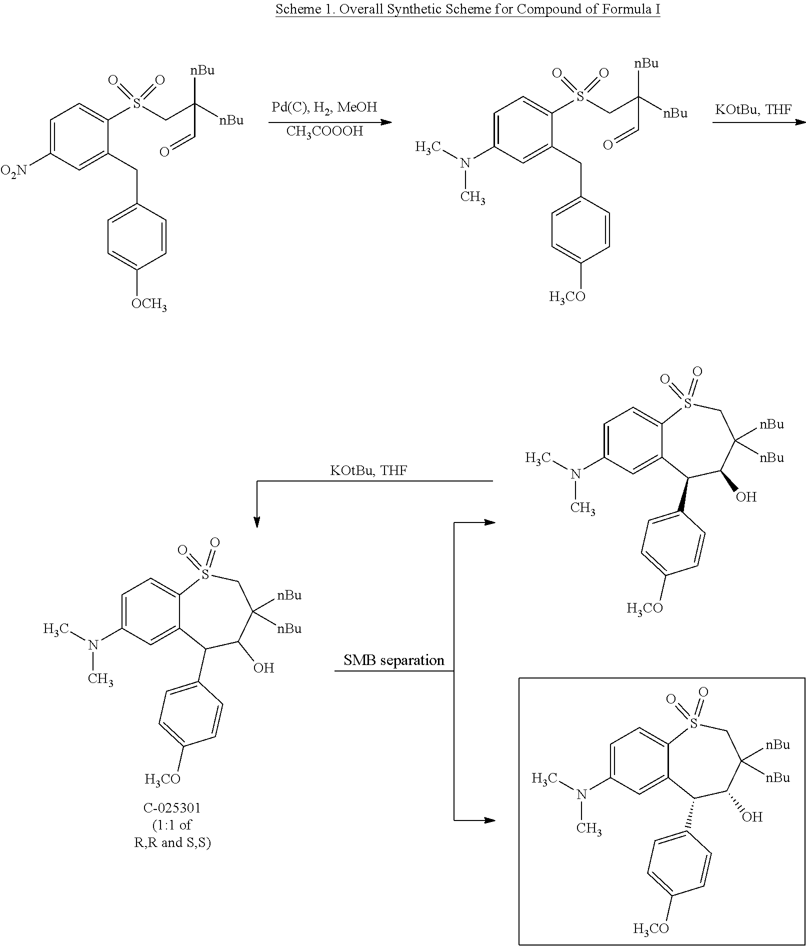

Compound of Formula I is synthesized by: (a) cyclizing acyclic precursor 2-butyl-2-(((4-(dimethylamino)-2-(4-methoxybenzyl)phenyl)sulfonyl)methypheptanal with potassium tert-butoxide (KOtBu) in tetrahydrofuran (THF) to generate a racemic mixture of 1:1 R,R and S,S stereoisomers; and (b) separating the R,R and S,S stereoisomers with Simulated Moving Bed (SMB) chromatography to obtain isolated R,R stereoisomer of Formula I, as shown in Scheme 1.

In another embodiment, the racemic mixture of 1:1 R,R and S,S isomers is generated by the recycling and racemization of the undesired S,S stereoisomer with KOtBu in THF in Scheme 1.

In some embodiments, the mobile phase used in the SMB conditions an inert solvent such as acetone, methyltetrahydrofuran (MeTHF), dichloromethane (DCM), pyridine, chlorobenzene (PhCl), methyl acetate (MeOAc), ethyl acetate (EtOAc), benzene, toluene, methyl tert-butyl ether (MtBE) and n-heptane.

In another embodiments, the mobile phase used in the SMB conditions were 70/30 n-heptane:DCM, 70/30 PhCl:n-heptane, and 90/10 toluene:n-heptane.

In some embodiments, the immobilized chiral stationary phases (CSP) were Chiralpak IA, Chiralpak OD-I(IB), Chiralpak IC, Chiralpak ID, Chiralpack IE, Chiralpak IF, Regis Whelk, O-2, Regis ULMO, Regis DACH-DMB, Regis Burke, Shiseido Ceramospher RU-1, and Shiseido Ceramospher RU-2.

In some embodiments, crystalline Form A of compound of Formula I was prepared from solvents such as isopropanol, ethanol (EtOH), methanol (MeOH), ACN, acetone, methyltetrahydrofuran (MeTHF), dichloromethane (DCM), pyridine, chlorobenzene (PhCl), methyl acetate (MeOAc), ethyl acetate (EtOAc), benzene, toluene, methyl tert-butyl ether (MtBE), n-heptane, 70/30 n-heptane:DCM, 70/30 PhCl:n-heptane, and 90/10 toluene:n-heptane. In a preferred embodiment, the crystalline Form A of compound of Formula I was prepared from nonprotic solvents.

In some embodiments, purified and crystalline compound of Formula I comprises about ≤0.30% to about ≤0.50% of the S,S stereoisomer.

Production of compound of Formula I utilized the Simulated Moving Bed (SMB) chromatography technique to separate the desired enantiomer from the racemic mixture. SMB is known for separating racemic mixtures into the enantiomers. SMB separation cycles were typically carried out 2-4 times to provide sufficient quantities of compound of Formula I for subsequent process stages. This process produced compound of Formula I with high purity. Other known chiral separation techniques can also be implemented. A sample of compound of Formula I with chiral purity of 99.5% and achiral purity of 100% was obtained and characterized using various analytical techniques with results described in Table 2-1. The polymorphs were characterized by X-ray powder diffraction (XRPD), Differential Scanning calorimetry (DSC), Thermogravimetric Analysis (TGA), and Polarized Light Microscopy (PLM). The XRPD data result showed that compound of Formula I is crystalline prepared by Example 2.1 and was assigned Form A.

TABLE 2-1
Detailed Characterization Results

| Tests | Results |
|---|---|
| ¹H NMR, ¹³C NMR | The results are in agreement with the structure |
| 2D NMR | of Formula I |
| FT-IR | |
| HRMS | |
| EA (CHNS) | |
| UV | The maximum absorption wavelength was 196 nm, |
| | 228 nm and 284 nm |
| XRPD | Crystalline, Form A |
| TGA | 0.1% weight loss up to 150° C. |
| DSC | One endothermic peak at 130.7° C. (onset) |
| DVS | Non-hygroscopic, and no form transition observed |
| | before and after DVS test |

### 2.2 Stability Studies on Compound of Formula I

Long-term and accelerated stability studies were performed as below using the compound of Formula I prepared as described in Example 2.1.

TABLE 2-2
Stability Studies on Compound of Formula I

| | Time Point (Months) | | | | | | | |
|---|---|---|---|---|---|---|---|---|
| Storage Condition | 0 | 1 | 3 | 6 | 9 | 12 | 18 | 24 |
| 25° C. ± 2° C./60% RH ± 5% RH | X | — | X | X | X | X | X | X |
| 40° C. ± 2° C./75% RH ± 5% RH | X | X | X | X | — | — | — | — |

X = testing performed.

Tests include appearance, purity by HPLC, assay by HPLC, chiral purity by HPLC, and water by KF titration.

Available 12-month stability data at the long-term storage condition is presented in Table 2-3. In addition, the stressed stability in 95/5 (v/v) acetonitrile /isopropanol solutions was also evaluated (e.g., during the SMB separation and successive work-up). Samples were stored under two different conditions over a period of 10 days: exposed to light at room temperature and in an oven at 60° C. The stability data is presented in Table 2-4. The purity of compound of Formula I (with regards to chemical and chiral purity) demonstrates adequate stability for both the SMB operation and subsequent work-up.

TABLE 2-3
Stability Data of Compound of Formula I- 25° C./60% RH

| | Acceptance | | Results | | | | | | | |
|---|---|---|---|---|---|---|---|---|---|---|
| Test | Criteria | | 0 | | 3 | | 6 | | 12 | |
| Appearance | White to yellow | | Off-white | | Off-white | | Off-white | | Off-white | |
| (Color, | solid | | solid | | solid | | solid | | solid | |
| morphology) | | | | | | | | | | |
| Appearance | Report result | | No further | | No further | | No further | | No further | |
| (Further | | | observations | | observations | | observations | | observations | |
| observations) | | | | | | | | | | |
| Identity by 1H | Conforms to | | Conforms | | Conforms | | Conforms | | Conforms | |
| NMR | Reference | | | | | | | | | |
| Water by KF | ≤1.0% | w/w | <0.1% | w/w | <0.1% | w/w | <0.1% | w/w | <0.1% | w/w |
| Titration | | | | | | | | | | |
| HPLC Purity - | ≥99.0% | a/a | 100.0% | a/a | 100.0% | a/a | 100.0% | a/a | 100.0% | a/a |
| Chiral | | | | | | | | | | |
| HPLC Purity | ≥99.0% | a/a | 99.6% | a/a | 99.9% | a/a | 99.8% | a/a | 99.8% | a/a |
| HPLC Assay | ≥90.0% | w/w | 95.5% | w/w | 96.3% | w/w | 96.4% | w/w | 96.5% | w/w |

TABLE 2-4
Heat and Light Stressed Stability Data
for Compound of Formula I in Solution

| Fraction | |
|---|---|
| Compound of Formula I | |
| Temperature | |
| RT (light) | T = 60° C. |

Purity (% a/a)

| | Chiral | Chemical | Chiral | Chemical |
|---|---|---|---|---|
| Day 0 | 99.6 | 99.4 | 99.7 | 99.3 |
| Day 3 | 99.6 | 99.4 | 99.5 | 98.7 |
| Day 7 | 99.7 | 99.4 | 99.8 | 98.5 |
| Day 10 | 99.4 | 99.4 | 99.7 | 98.5 |

### 2.3 Form A of Compound of Formula I

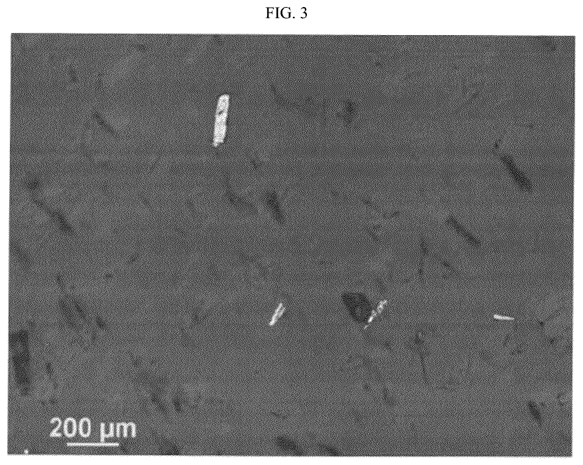

The XRPD, TGA/DSC, and PLM characterizations with the same parameters described above were performed using the compound of Formula I prepared as described in Example 2.1. Form A crystalline of compound of Formula I was identified, based on the XRPD patterns shown in FIG. 1. As shown by TGA and DSC data in FIG. 2, there was no weight loss before decomposition, which starts at approximately 200° C. A melting endotherm with onset temperature of 130.8° C. (onset) and enthalpy of 47 J/g was observed. Additionally, an endotherm like event was observed at 255° C., most likely caused by pressure buildup due to the decomposition of the sample. A polarizing light microscopy (PLM) examination revealed that material consists of rod-shaped crystals (FIG. 3).

### 2.4 Form B of Compound of Formula I

Form B was obtained from anti-solvent addition experiment with ACN/water from Form A. Form B was observed only as a wet cake, which convers back to Form A when air-dried. The XRPD, TGA/DSC, and PLM characterizations with the same parameters described above were performed. FIG. 4 shows XRPD of the wet cake of Form B. TGA of the Form B was also obtained and showed a weigh loss of 45.09% up to 100° C. (FIG. 5). The weight loss started immediately with heat applied and it stopped 75° C. The wet cake sample was air-dried overnight, the XRPD of the air-dried cake showed that it was converted back to Form A.

### Example 3. Polymorph Screening of Compound of Formula I

The approximate solubility of compound of Formula I (starting material, prepared as described above) was measured at RT in 20 different solvents. Approximately 2 mg of starting material were added into a 3-mL glass vial. Solvents were then added step wise into the vials until the solids were dissolved or a total volume of 1 mL was reached (Note: this is not thermodynamic solubility estimation, vortex and sonication were applied in between each solvent to accelerate the dissolution). Results summarized in Table 3-1 were used to guide the solvent selection for the polymorph screening. Beginning with the starting material, polymorph screening experiments were set up using methods of Slurry at different temperatures, Slow Cooling, Liquid and Solid Vapor Diffusion, Slow Evaporation, Anti-Solvent Addition and Ionic Liquid. The methods utilized, and crystal forms identified are summarized in Table 3-2 (Type A and Form A are used interchangeably and Type B and Form B are used interchangeably).

TABLE 3-1
Approximate solubility of starting material at RT

| Solvent (v:v) | Solubility (mg/mL)* |
|---|---|
| Acetone | 54.0 < S |
| ACN | 44.0 < S |
| n-Butyl Acetate | 56 < S |
| Cyclohexane | S < 2 |
| DCM | 58.0 < S |
| DMF | 48 < S |
| DMSO | 50.0 < S |
| EtOAc | 40.0 < S |
| EtOH | 27 < S < 54.0* |
| H2O | S < 2.3 |
| n-Heptane | S < 2.8 |
| IPA | 7.3 < S < 22 |
| IPAc | 24 < S < 48 |
| MeOH | 29 < S < 58 |
| MeTHF | 56.0 < S |
| MIBK | 50.0 < S |
| MTBE | 9.3 < S < 28 |
| NMP | 46.0 < S |
| Pentane | S < 2.7 |
| Toluene | 54.0 < S |

*approximate solubility (S) is reported as a range; low end < S < high end

TABLE 3-2
Summary of polymorph screening experiments

| | No. of | |
|---|---|---|
| | Exper- | |
| Method | iments | Crystal Type |
| Slurry RT | 15 | Type A |
| Slurry 65° C. | 15 | Type A |
| Anti Solvent Addition | 11 | Type A, Type B, Gel, Amorphous |
| Solid Vapor Diffusion | 10 | Type A, Gel, Amorphous |
| Liquid Vapor Diffusion | 12 | Type A, Gel |
| Slow Evaporation | 10 | Type A, Gel, Clear, Amorphous |
| Ionic Liquid | 10 | Type A, Gel, Clear, Amorphous |
| Slow Cooling | 10 | Type A, Gel, Clear, Amorphous |
| Total | 93 | Type A, Clear, Amorphous |

### 3.1 Slurry at RT

A total of 15 Slurry at Room Temperature experiments were carried out. For each experiment, approximately 20 mg of starting material were weighed in a 2 mL glass vial, and 0.2 mL of corresponding solvent were added to form a suspension. This suspension was magnetically stirred for 3 days at room temperature (25° C.). After three days, the suspension was taken out of the vial for a wet cake 4 min XRPD analysis.

Summary of experiments in Table 3-3 below shows the solvents used for each experiment, its corresponding volume, and indicates that Form A (i.e. Type A) was the only solid form obtained in these experiments.

TABLE 3-3
Summary of slurry experiments at RT

| Experiment ID | Solvent (v:v) | Volume | Solid Form |
|---|---|---|---|
| 6022370-03-A1 | Cyclohexane | 200 uL | Type A |
| 6022370-03-A2 | H₂O | 200 uL | Type A |
| 6022370-03-A3 | n-Heptane | 200 uL | Type A |
| 6022370-03-A4 | IPA | 200 uL | Type A |
| 6022370-03-A5 | MTBE | 200 uL | Type A |
| 6022370-03-A6 | Pentane | 200 uL | Type A |
| 6022370-03-A7 | Cyclohexane/Acetone (3:2) | 200 uL | Type A |
| 6022370-03-A8 | Cyclohexane/DCM (3:2) | 200 uL | Type A |
| 6022370-03-A9 | H₂O/DMSO (3:2) | 200 uL | Type A |
| 6022370-03-A10 | H₂O/EtOH (3:2) | 200 uL | Type A |
| 6022370-03-A11 | n-Heptane/Toluene (3:2) | 200 uL | Type A |
| 6022370-03-A12 | IPA/Toluene (3:2) | 200 uL | * |
| 6022370-03-A13 | MTBE/DMSO (3:2) | 200 uL | * |
| 6022370-03-A14 | Pentane/DCM (3:2) | 200 uL | Type A |
| 6022370-03-A15 | Pentane/acetone (3:2) | 200 uL | Type A |

*Did not form a suspension and dissolved after 100 mg or more added.

### 3.2 Slurry at 65° C.

A total of 15 Slurry at 65° C. experiments were carried out. For each experiment, approximately 20 mg of starting material were weighed in a 2 mL glass vial, and 0.2 mL of corresponding solvent were added to form a suspension. This suspension was magnetically stirred for 3 days at 65° C.

After three days, the suspension was scooped out of the vial for a wet cake 4 min XRPD analysis. Summary of experiments in Table 3-4 below shows the solvents used for each experiment, its corresponding volume, and indicates that Form A (i.e. Type A) was the only solid form obtained in these experiments.

TABLE 3-4
Summary of slurry experiments at 65° C.

| Experiment ID | Solvent (v:v) | Volume | Solid Form |
|---|---|---|---|
| 6022370-04-A1 | Cyclohexane | 200 uL | Type A |
| 6022370-04-A2 | H20 | 200 uL | Type A |
| 6022370-04-A3 | n-Heptane | 200 uL | Type A |
| 6022370-04-A4 | IPA | 200 uL | * |
| 6022370-04-A5 | MTBE | 200 uL | * |
| 6022370-04-A6 | Pentane | 200 uL | Type A |
| 6022370-04-A7 | Cyclohexane/Toluene (3:2) | 200 uL | Type A |
| 6022370-04-A8 | Cyclohexane/DCM (3:2) | 200 uL | * |
| 6022370-04-A9 | n-Heptane/Butyl Acetate (3:2) | 200 uL | Type A |
| 6022370-04-A10 | n-Heptane/Toluene (3:2) | 200 uL | Type A |
| 6022370-04-A11 | IPA/Acetone (2:1) | 200 uL | Type A |
| 6022370-04-A12 | IPA/ DMF (2:1) | 200 uL | * |
| 6022370-04-A13 | MTBE/ACN (2:1) | 200 uL | Type A |
| 6022370-04-A14 | MTBE/DMSO (2:1) | 200 uL | Type A |
| 6022370-04-A15 | MTBE/Butyl Acetate (2:1) | 200 uL | Type A |

*Did not form a suspension; dissolved after 100 mg or more of starting material added.

### 3.3 Anti-Solvent Addition

A total of 11 anti-solvent addition experiments were carried out. For each experiment, approximately 20 mg of starting material were weighed in a 20 ml glass vial then dissolved in 0.2 mL of corresponding solvent in order to obtain a clear solution. These solutions were magnetically stirred at room temperature (25° C.) while corresponding anti solvent was continuously added dropwise (every one second) until crystallization was observed. The obtained precipitates were scooped out of the vial for wet cake 4 min XRPD analysis. For the samples in which crystallization was not observed, the addition of antisolvent ceased at 10 mL, and these samples were moved to 5° C. for 3 days. The resulting solids were scooped out of the vial for wet cake 4 min XRPD analysis. Summary of experiments in Table 3-5 below shows the solvents used for each experiment, its corresponding volume, and indicates that Form A (i.e. Type A), Form B (i.e. Type B), Gel and amorphous were obtained in these experiments.

TABLE 3-5
Summary of anti-solvent addition experiments

| Experiment ID | Solvent | Volume | Anti-solvent | Volume | Solid Form |
|---|---|---|---|---|---|
| 6022370-05-A1 | Acetone | 200 uL | Cyclohexane | 10 mL | Gel** |
| 6022370-05-A2 | Acetone | 200 uL | Water | 300 uL | Amorphous |
| 6022370-05-A3 | Toluene | 200 uL | Cyclohexane | 10 mL | Type A** |
| 6022370-05-A4 | Toluene | 200 uL | n-Heptane | 10 mL | Type A |
| 6022370-05-A5 | Toluene | 200 uL | MTBE | 10 mL | No solids** |
| 6022370-05-A6 | ACN | 200 uL | IPA | 10 mL | Amorphous** |
| 6022370-05-A7 | ACN | 200 uL | Water | 2 mL | Type B →Type A*** |
| 6022370-05-A8 | DCM | 200 uL | Cyclohexane | 10 mL | Type A** |
| 6022370-05-A9 | DCM | 200 uL | IPA | 10 mL | Type A* |
| 6022370-05-A10 | N-Butyl | 200 uL | Ipa | 10 mL | Amorphous* |
| | Acetate | | | | |
| 6022370-05-A11 | Ethyl | 200 uL | n-Heptane | 10 mL | Type A |
| | acetate | | | | |

*Samples moved to 5° C. for 3 days.
**Samples moved to evaporation at RT after 5° C. for 3 days.
***Wet cake shows a new form, named Type B. However, it converted to Type A after air dried.

### 3.4 Solid Vapor Diffusion

A total of 10 Solid Vapor Diffusion experiments were carried out. For each experiment, approximately 20 mg of starting material were weighed in a 4 mL glass vial which was not sealed with a cap and was placed inside a 20-mL vial that was filled with approximately 3 mL of volatile solvent. The 20 mL vial was then sealed with a cap and kept at RT for 7 days, allowing the solvent vapor to interact with the starting material inside of the 4 mL vial. After 7 days, the solids were taken out of the vial for wet cake 4 min XRPD analysis.

Vials in which the starting material fully converted to liquid after interacting with the solvent vapors, were set for evaporation for 5 days. After this period, the resulting solids were taken out of the vial for wet cake 4 min XRPD analysis.

Summary of experiments in Table 3-6 below shows the solvents used for each experiment, its corresponding volume, and indicates that Form A (i.e. Type A), gel and amorphous were the solid forms obtained in these experiments.

TABLE 3-6
Summary of solid vapor diffusion experiments

| Experiment ID | Solvent | Volume | Solid Form |
|---|---|---|---|
| 6022370-06-A1 | Acetone | 3 mL | No solids* |
| 6022370-06-A2 | ACN | 3 mL | Type A |
| 6022370-06-A3 | n-Butyl Acetate | 3 mL | Type A |
| 6022370-06-A4 | DCM | 3 mL | Type A |
| 6022370-06-A5 | DMF | 3 mL | Gel* |
| 6022370-06-A6 | DMSO | 3 mL | Amorphous |
| 6022370-06-A7 | EtOAc | 3 mL | Type A |
| 6022370-06-A8 | Me0H | 3 mL | Type A |
| 6022370-06-A9 | NMP | 3 mL | Gel* |
| 6022370-06-A10 | MIBK | 3 mL | Type A |

*Samples in which the starting material fully converted to liquid were set for evaporation for 5 days. After this period

### 3.5 Liquid Vapor Diffusion

A total of 12 liquid vapor diffusion experiments were carried out. For each experiment, approximately 20 mg of starting material were weighed in a 4 mL glass vial and dissolved in correspondent solvent to obtain a clear solution. This vial was not sealed and was placed inside a 20-mL vial that was filled with approximately 3 mL of volatile anti solvent. The 20 mL vial was then sealed with a cap and kept at RT for 7 days, allowing the anti-solvent vapor to interact with the solution inside of the 4 mL vial. After 7 days, the solids were taken out of the vial for wet cake 4 min XRPD analysis. Vials in which no solids were observed, were set for evaporation for 5 days, and after this period, the resulting solids were taken out of the vial for wet cake 4 min XRPD analysis.

Summary of experiments in Table 3-7 below shows the solvents and anti-solvents used for each experiment, its corresponding volume, and indicates that Form A (i.e. Type A) was the only solid form obtained in these experiments.

TABLE 3-7
Summary of liquid vapor diffusion experiments

| Experiment ID | Solvent | Volume | Anti-solvent | Volume | Solid Form |
|---|---|---|---|---|---|
| 6022370-07-A1 | n-Butyl acetate | 100 ul | cyclohexane | 3 mL | Type A |
| 6022370-07-A2 | | | heptane | | Type A |
| 6022370-07-A3 | | | IPA | | Type A * |
| 6022370-07-A4 | | | MTBE | | Type A * |
| 6022370-07-A5 | | | Pentane | | Type A |
| 6022370-07-A6 | EtOAc | | Heptane | | Type A |
| 6022370-07-A7 | | | MTBE | | Type A* |
| 6022370-07-A8 | | | Pentane | | Type A |
| 6022370-07-A9 | Toluene | | Cyclohexane | | Gel* |
| 6022370-07-A10 | | | Pentane | | Type A |
| 6022370-07-A11 | | | MTBE | | Type A* |
| 6022370-07-A12 | | | IPA | | Type A |

*No solids observed, so samples were transferred for evaporation.

### 3.6 Slow Evaporation

A total of 10 slow evaporation experiments were carried out. For each experiment, approximately 20 mg of starting material were weighed in a 2 mL vial then dissolved in 0.2 mL of corresponding solvent to obtain a clear solution. The vials were then covered by Parafilm® with 3 pinholes and subjected to evaporation at room temperature for 7 days. If after this period no solids were observed, the samples were moved to 5° C. for 3 days to induce precipitation.

The resulting solids were taken out of the vial for wet cake 4 min XRPD analysis.

Summary of experiments in Table 3-8 below shows the solvents used for each experiment, its corresponding volume, and indicates that Form A (i.e. Type A), Gel and Amorphous were obtained in these experiments.

TABLE 3-8
Summary of slow evaporation experiments

| | Solvent | | Solid |
|---|---|---|---|
| Experiment ID | (v:v) | Volume | Form |
| 6022370-08-A1 | DMF | 100 uL | Gel |
| 6022370-08-A2 | MeTHF | 100 uL | Gel |
| 6022370-08-A3 | MIBK | 100 uL | Gel |
| 6022370-08-A4 | NMP | 100 uL | Clear solution |
| 6022370-08-A5 | Toluene | 100 uL | Type A |
| 6022370-08-A6 | DCM | 100 uL | Amorphous |
| 6022370-08-A7 | Acetone | 100 uL | Amorphous |
| 6022370-08-A8 | ACN | 100 uL | Type A |
| 6022370-08-A9 | n butyl acetate | 100 uL | Type A |
| 6022370-08-A10 | DMSO | 100 uL | Gel |

### 3.7 Ionic Liquid

A total of 10 ionic liquid experiments were carried out. For each experiment, approximately 20 mg of starting material were weighed in a 2 mL glass vial then dissolved in 0.1 mL of corresponding solvent to obtain a clear solution. About 5 mg (25 wt % of starting material) of ionic liquid 1-Butyl-3-methyl imidazolium chloride were added into each glass vial. All the samples were subjected to evaporation at room temperature to induce precipitation for 7 days. No solids were observed after this period, so all samples were moved to 5° C. for 3 days.

The resulting solids were taken out of the vial for dry cake 4 min XRPD analysis. Summary of experiments in Table 3-9 below shows the solvents used for each experiment, its corresponding volume, and indicates that Form A (i.e. Type A), Gel and Amorphous were obtained.

TABLE 3-9
Summary of Ionic Liquid crystallization experiments

| | Solvent | | Ionic | | Solid |
|---|---|---|---|---|---|
| Experiment ID | (v/v) | Volume | Liquid | Weight | Form |
| 6022370-09-A1 | DMF | 100 uL | 1-Butyl-3- | 3 mg | Gel |
| 6022370-09-A2 | MeTHE | 100 uL | methyl | | Gel |
| 6022370-09-A3 | MIBK | 100 uL | imidazolium | | Gel |
| 6022370-09-A4 | NMP | 100 uL | chloride | | Clear |
| 6022370-09-A5 | Toluene | 100 uL | | | Gel |
| 6022370-09-A6 | DCM | 100 uL | | | Type A |
| 6022370-09-A7 | Acetone | 100 uL | | | Amorphous |
| 6022370-09-A8 | EtOH | 100 uL | | | Type A |
| 6022370-09-A9 | IPAc | 100 uL | | | Amorphous |
| 6022370-09-A10 | DMSO | 100 uL | | | Amorphous |

Note:
No solids formed in any of the vials after 7 days, so all of them were moved to 5° C. for 3 days.

### 3.8 Slow Cooling

A total of 10 slow cooling experiments were carried out. For each experiment, approximately 20 mg of starting material were weighed in a 2 mL glass vial then 0.1 mL of correspondent solvent were added to form saturated solutions. Those solutions were then cooled from 50° C. to 5° C. over a period of 7.5 hrs inside of an incubator. After this period, all solutions were clear (no solids). They were then set to slow evaporate at room temperature for 5 days. No solids were observed after 5 days, so the samples were moved to −14° C. for 3 days. After this period, the resulting solids were taken out of the vial for dry cake 4 min XRPD analysis.

Summary of experiments in Table 3-10 below shows the solvents used for each experiment, its corresponding volume, and indicates that Form A (i.e. Type A), Gel and Amorphous were obtained in these experiments.

TABLE 3-10
Summary of slow cooling experiments

| Experiment ID | Solvent (v:v) | Volume | Solid Form |
|---|---|---|---|
| 6022370-10-A1 | EtOH | 100 uL | Type A |
| 6022370-10-A2 | IPAc | 100 uL | Gel |
| 6022370-10-A3 | MeOH | 100 uL | Type A |
| 6022370-10-A4 | MTBE | 100 uL | Type A |
| 6022370-10-A5 | MIBK | 100 uL | Gel |
| 6022370-10-A6 | ACN | 100 uL | Type A |
| 6022370-10-A7 | Acetone | 100 uL | No solids |
| 6022370-10-A8 | NMP | 100 uL | Clear solution |
| 6022370-10-A9 | Toluene | 100 uL | Amorphous |
| 6022370-10-A10 | MeTHF | 100 uL | Type A |

As described above, polymorph screening experiments were set up using methods of Slurry at different temperatures, Slow Cooling, Liquid and Solid Vapor Diffusion, Slow Evaporation, Anti-Solvent Addition, and Ionic Liquid. Two types, Form A (anhydrate) and Form B (weak solvate) were obtained from the experiments. Form B was obtained from the wet cake of Anti-solvent Addition experiment with ACN/water; however, the dry cake converted back to Form A.

Further investigation was performed for Form B to better understand the phase origin. Form B was successfully remade with the same method (anti-solvent addition with ACN/water), the centrifuged wet cake was quickly tested by TGA and resulted in 45% weight loss. The air-dried cake again converted back to Form A. Since none of the other experiments involving water or acetonitrile produced Form B, Form B is either mixed very labile acetonitrile/water solvate/hydrate, or a metastable polymorph.

Other than Form B (i.e., Type B), no additional polymorph was observed during the rest of the screening. However, Form B (i.e., Type B) can easily convert back to Form A (i.e. Type A) upon drying, therefore, it is concluded that Form A (i.e. Type A) is the most stable form under the conditions tested in this study and is recommended for further development.

### Example 4. Synthesis of Compound of Formula III

The synthetic scheme for the manufacture of maralixibat chloride (Formula III), beginning with compound of Formula I, is shown below. Table 4-1 shows the abbreviations for the reagents used in the synthesis.

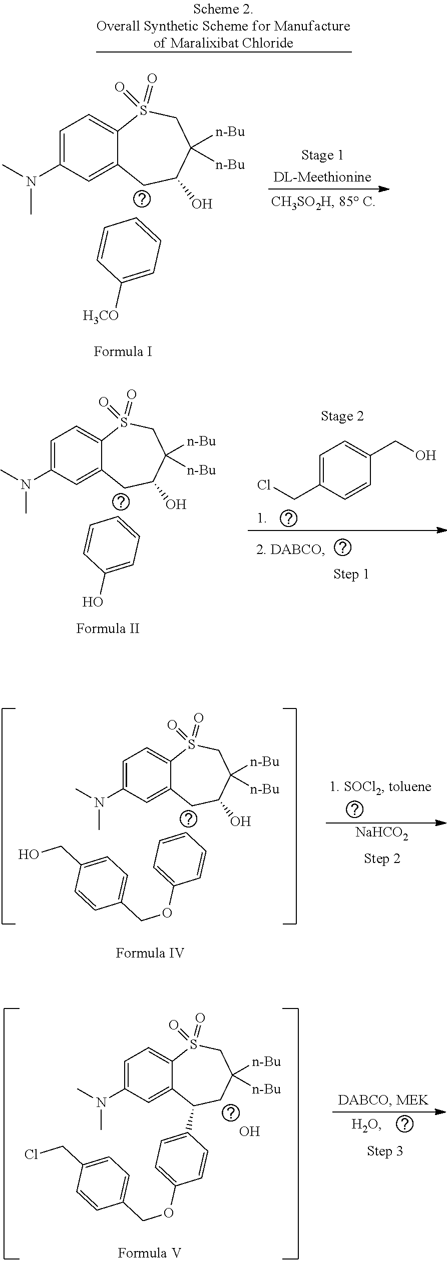

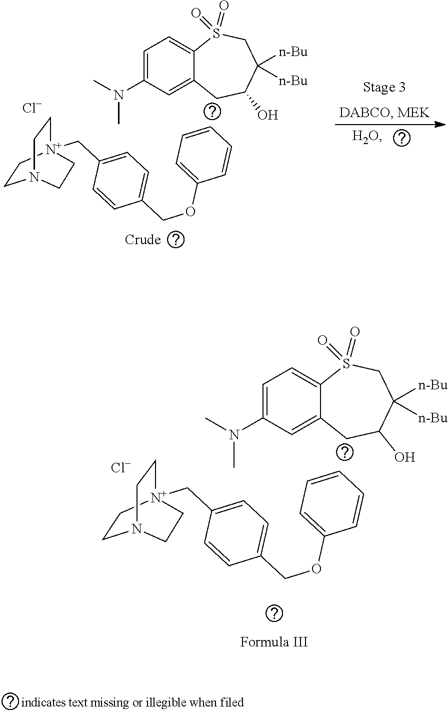

TABLE 1-1
List of Abbreviations

| | |
|---|---|
| CH₃SO₃H | Methanesulfonic acid |
| CMBA | 4-(Chloromethyl) benzyl alcohol |
| MIBK | Methyl isobutyl ketone |
| K₃PO₄ | Potassium phosphate tribasic |
| DABCO | 1,4-diazabicyclo[2.2.2]octane |
| SOCl₂ | Thionyl chloride |
| NaHCO₃ | Sodium bicarbonate |
| MEK | Methyl ethyl ketone |

The synthesis of compound of Formula III is a three stage (four synthetic steps) linear synthesis process. Formula I is demethylated to Formula II which is telescoped through a three (3) step process to crude maralixibat. The intermediates are not isolated. Crude maralixibat is crystallized to Maralixibat. The typical batch size of the maralixibat drug substance varies based on commercial demand and is expected to be ˜15-20 kg. Some of the examples provided herein were implemented at a smaller scale (˜5 kg). All reactions are stirred under a nitrogen atmosphere unless otherwise indicated.

### Stage 1—Synthesis of Compound of Formula II from Compound of Formula I

A reaction vessel is charged with methanesulfonic acid, followed by methionine and compound of Formula I. The mixture is heated and an in-process check (IPC) by HPLC is performed to ensure that the conversion is ≥98%. The reaction mixture is diluted with water and extracted with methyl isobutyl ketone (MIBK). The aqueous phase is back extracted with MIBK. The combined organic layer is washed with aqueous sodium bicarbonate followed by a water wash. The organic phase is filtered through carbon and distilled to the target volume. The resulting slurry is diluted with heptane to complete the precipitation. The mixture is cooled and the product is collected by filtration and washed with a mixture of heptane and MIBK. The product is dried under reduced pressure to give compound of formula II in approximately 90% yield and 99% purity.

### Stage 2—Synthesis of Crude Maralixibat from Compound of Formula II

### Step 1—Synthesis of Compound of Formula IV Compound of Formula II

A reaction vessel is charged with compound of Formula II, potassium phosphate, 4-(Chloromethyl) benzyl alcohol (CMBA, C-003838) and acetone and heated to reflux. An in-process check is performed using HPLC analysis to ensure that conversion to compound of Formula IV is ≥97%.

1,4-Diazabicyclo-[2.2.2] octane is added to the reaction mixture to consume any unreacted CMBA reagent. After confirming that CMBA content is ≤0.05% by an in-process check, the mixture is cooled and diluted with toluene and the organic layer is washed with water. The layers are separated and the organic phase is concentrated under reduced pressure to the target volume to give crude (4R,5R)-3,3-dibutyl-5-[4-[[4-(hydroxymethyl) phenyl]methoxy]phenyl]-7-(dimethylamino)-2,3,4,5-tetrahydro-1-benzothiepin-4-ol 1,1-dioxide (Formula IV) in toluene. The organic solution containing Formula IV is used in the next step without further purification.

Step 2—Synthesis of Compound of Formula V from Compound of Formula IV

The organic solution containing compound of Formula IV from the previous step is reacted with thionyl chloride diluted in toluene while maintaining the temperature between 20° C.±5° C. The solution is stirred and an in-process check is performed to ensure that the conversion to Formula V has reached ≥99%. The reaction mixture is washed with water. After phase separation, 7.4% sodium bicarbonate solution is used to wash the organic phase, followed by another two water washes after each phase separation. The organic phase is concentrated under reduced pressure to the target volume. (4R, 5R)-3,3-dibutyl-5-(4-((4-(chloromethyl)benzypoxy)phenyl)-7-(dimethylamino)-4-hydroxy-2,3,4,5-tetrahydrobenzo[b]thiepine 1,1-dioxide (Formula V) is used in the next step without further purification.

Step 3—Synthesis of Crude Maralixibat from Compound of Formula V

The solution containing compound of Formula V is added to a heated solution of DABCO in MEK/water containing C-025325 seed. An HPLC check is performed to ensure that the conversion is ≥99.5%. After completion of the reaction, MEK is added to complete the precipitation of the product. The suspension is cooled, and the solid is collected by filtration and dried to provide the crude maralixibat. An overall yield of approximately 90% over the three steps is obtained.

### Stage 3—Purification of Crude Maralixibat

Crude maralixibat is dissolved in MEK/water/DABCO, seeded with C-025325 and heated. To the solution, MEK is added to crystallize the product. The suspension is cooled, filtered and dried to form the recrystallized maralixibat in approximately 90% yield and >99% purity and 100% chiral purity.

### Example 4. Characterization of Compound of Formula II

### 4.1 Form X of Compound of Formula II

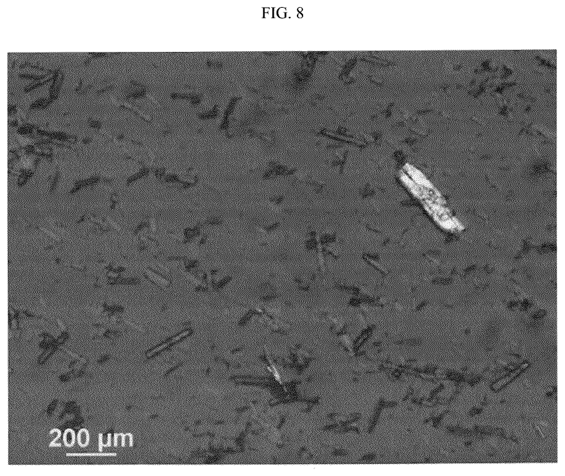

The XRPD, TGA/DSC, and PLM characterizations with the same parameters described above were performed using the sample prepared according to stage 1 of Example 4 from compound of Formula I prepared as described in Example 2.1. Form X crystalline of compound of Formula II was identified, based on the XRPD patterns shown in FIG. 6. As shown by TGA and DSC data in FIG. 7, the material is anhydrous, and melts at 216.1° C., as determined by the onset of the melting endotherm. Rod shaped crystals were observed by PLM (FIG. 8). Based on the thermal data, the starting material was determined to be an anhydrate.

### Example 5. Polymorph Screening of Compound of Formula II

Polymorph Screening experiments were set up using methods of Slurry at different temperatures, Slow Cooling, Liquid and Solid Vapor Diffusion, Slow Evaporation, Anti-Solvent Addition, and Ionic Liquid. However, the data showed that in all these conditions the only crystalline form is Form X. No new polymorph was observed during the screening.

The approximate solubility of compound of Formula II (starting material) was determined at RT in 20 different solvents. Approximately 2 mg of the material were added into a 2-mL glass vial. Solvents were then added step into the vials until the solids were dissolved or a total volume of 1 mL was reached (Note: this is not a thermodynamic solubility estimation; vortex and sonication were applied in between each solvent addition to accelerate the dissolution). Results summarized in Table 5-1 were used to guide the solvent selection for the polymorph screening. Beginning with the starting material, polymorph screening experiments were set up using methods of Slurry at different temperatures, Slow Cooling, Liquid and Solid Vapor Diffusions, Slow Evaporation, Anti-Solvent Addition, and Ionic Liquid. The methods utilized, and crystal forms identified are summarized in Table 5-2. No new polymorphs were observed during the screening. Type X and Form X are used interchangeably.

TABLE 5-1
Approximate solubility of starting material at RT

| Solvent (v:v) | Solubility (mg/mL)* |
|---|---|
| Acetone | 26 < S < 52 |
| ACN | 2.9 < S < 9.7 |
| n-Butyl Acetate | 8.3 < S < 25 |
| Cyclohexane | S < 2 |
| DCM | 8.7 < S < 26 |
| DMF | 40 < S |
| DMSO | 44 < S |
| EtOAc | 20 < S < 40 |
| EtOH | 2.7 < S < 9.3 |
| H2O | S < 2 |
| n-Heptane | S < 2 |
| IPA | 2.6 < S < 8.7 |
| IPAc | 7.7 < S < 23 |
| MeOH | 7.3 < S < 22 |
| MeTHF | 21 < S < 42 |
| MIBK | 21 < S < 43 |
| MTBE | 2.4 < S < 8 |
| NMP | 44 < S |
| Pentane | S < 2.7 |
| Toluene | S < 2.8 |

*Approximate solubility (S) is reported as a range; low end < S < high end

TABLE 5-2
Summary of polymorph screening experiments

| Method | No. of Experiments | Crystal Type |
|---|---|---|
| Slurry RT | 15 | Type X |
| Slurry 65° C. | 15 | Type X |
| Anti-Solvent Addition | 8 | Type X |
| Solid Vapor Diffusion | 10 | Type X |
| Liquid Vapor Diffusion | 8 | Type X, Amorphous |
| Slow Evaporation | 10 | Type X, Amorphous |
| Ionic Liquid | 10 | Type X, Amorphous |
| Slow Cooling | 10 | Type X |
| Total | 86 | Type X, Amorphous |

### 5.1 Slurry at RT

A total of 15 Slurry at Room Temperature experiments were carried out. For each experiment, approximately 20 mg of starting material were weighed in a 2 mL glass vial, and 0.2 mL of corresponding solvent were added to Form X (i.e., Type X) suspension. This suspension was magnetically stirred for 3 days at room temperature (25° C.). After three days, the suspension was taken out of the vial for a 4 min XRPD analysis of the wet cakes.

Summary of experiments in Table 5-3 below shows the solvents used for each experiment, its corresponding volume, and indicates that Form X (i.e., Type X) was the only solid form obtained in these experiments.

TABLE 5-3
Summary of slurry experiments at RT

| Experiment ID | Solvent (v:v) | Volume | Solid Form |
|---|---|---|---|
| 6022369-03-A1 | Cyclohexane | 200 uL | Type X |
| 6022369-03-A2 | MTBE | 200 uL | Type X |
| 6022369-03-A3 | EtOH | 200 uL | Type X |
| 6022369-03-A4 | H₂O | 200 uL | Type X |
| 6022369-03-A5 | n-Heptane | 200 uL | Type X |
| 6022369-03-A6 | IPA | 200 uL | Type X |
| 6022369-03-A7 | Pentane | 200 uL | Type X |
| 6022369-03-A8 | Toluene | 200 uL | Type X |
| 6022369-03-A9 | Cyclohexane/ | 200 uL | Type X |
| | Acetone 3:2 | | |
| 6022369-03-A10 | H₂O/DMF 2:1 | 200 uL | Type X |
| 6022369-03-A11 | n-Heptane/Ethyl | 200 uL | Type X |
| | Acetate 3:2 | | |
| 6022369-03-A12 | Pentane/ | 200 uL | Type X |
| | Acetone 3:2 | | |
| 6022369-03-A13 | Toluene/ | 200 uL | Type X |
| | DMSO 3:2 | | |
| 6022369-03-A14 | Toluene/ | 200 uL | Type X |
| | DMF 3:2 | | |
| 6022369-03-A15 | Butyl acetate/ | 200 uL | Type X |
| | ACN 1:1 | | |

### 5.2 Slurry at 65° C.

A total of 15 Slurry at 65° C. experiments were carried out. For each experiment, approximately 20 mg of starting material were weighed in a 2 mL glass vial, and 0.2 mL of corresponding solvent were added to Form X (i.e., Type X) suspension. This suspension was magnetically stirred for 3 days at 65° C. After three days, the suspension was scooped out of the vial for a 4 min XRPD analysis of the wet cakes. Summary of experiments in Table 5-4 below shows the solvents used for each experiment, its corresponding volume, and indicates that Form X (i.e., Type X) was the only solid form obtained in these experiments.

TABLE 5-4
Summary of slurry experiments at 65° C.

| Experiment ID | Solvent (v:v) | Volume | Solid Form |
|---|---|---|---|
| 6022369-04-A1 | Cyclohexane | 200 uL | Type X |
| 6022369-04-A2 | MTBE | 200 uL | Type X |
| 6022369-04-A3 | EtOH | 200 uL | Type X |
| 6022369-04-A4 | H₂O | 200 uL | Type X |
| 6022369-04-A5 | n-Heptane | 200 uL | Type X |
| 6022369-04-A6 | IPA | 200 uL | Type X |
| 6022369-04-A7 | Pentane | 200 uL | Type X |
| 6022369-04-A8 | Toluene | 200 uL | Type X |
| 6022369-04-A9 | Butyl acetate/ | 200 uL | Type X |
| | DCM 1:1 | | |
| 6022369-04-A10 | Cyclohexane/butyl | 200 uL | Type X |
| | acetate 1:1 | | |
| 6022369-04-A11 | EtOH/DMF 3:2 | 200 uL | Type X |
| 6022369-04-A12 | EtOH/DMSO 3:2 | 200 uL | Type X |
| 6022369-04-A13 | n-Heptane/ | 200 uL | Type X |
| | Acetone 3:2 | | |
| 6022369-04-A14 | H₂O/DMF 3:2 | 200 uL | Type X |
| 6022369-04-A15 | H₂O/DMSO 3:3 | 200 uL | Type X |

### 5.3 Anti-Solvent Addition

A total of 11 anti-solvent addition experiments were carried out. For each experiment, approximately 20 mg of starting material were weighed in a 20 mL glass vial then dissolved in 0.2 mL of corresponding solvent to obtain a clear solution. These solutions were magnetically stirred at room temperature (25° C.) while corresponding antisolvent was continuously added dropwise (every one second) until crystallization was observed. In these experiments, no crystallization was observed, the addition of antisolvent ceased at 10 mL, and all samples were moved to 5° C. for 3 days. The resulting solids were taken out of the vial for 4 min XRPD analysis of the wet cakes.

Summary of experiments in Table 5-5 below shows the solvents used for each experiment, its corresponding volume, and indicates that only Form X (i.e., Type X) was obtained in these experiments.

TABLE 5-5
Summary of anti-solvent addition experiments

| Experiment ID | Solvent | Volume | Anti-solvent | Volume | | Solid Form |
|---|---|---|---|---|---|---|
| 6022369-05-A1 | DMF | 200 uL | H₂O | 800 | uL | Type X* |
| 6022369-05-A2 | DMF | 200 uL | Toluene | 10 | mL | Type X* |
| 6022369-05-A3 | DMSO | 200 uL | H₂O | 800 | uL | Type X |
| 6022369-05-A4 | DMSO | 200 uL | Toluene | 10 | mL | Low crystalline* |
| 6022369-05-A5 | Acetone | 200 uL | n-Heptane | 6 | mL | Type X* |
| 6022369-05-A6 | Acetone | 200 uL | Pentane | 4 | mL | Type X |
| 6022369-05-A7 | EtOAc | 200 uL | n-Heptane | 6 | mL | Type X* |
| 6022369-05-A8 | EtOAc | 200 uL | Toluene | 10 | mL | Type X* |

*The samples were moved to 5° C. for 3 days.
Low crystalline: few reflection peaks but no amorphous halo was observed.

### 5.4 Solid Vapor Diffusion

A total of 10 Solid Vapor Diffusion experiments were carried out. For each experiment, approximately 20 mg of starting material were weighed in a 4 mL glass vial which was not sealed with a cap and was placed inside a 20-mL vial that was filled with approximately 3 mL of volatile solvent. The 20 mL vial was then sealed with a cap and kept at RT for 7 days, allowing the solvent vapor to interact with the starting material inside of the 4 mL vial. After 7 days, the solids were taken out of the vial for 4 min XRPD analysis of the wet cakes.

Summary of experiments in Table 5-6 below shows the solvents used for each experiment, its corresponding volume, and indicates that only Form X (i.e., Type X) was obtained in these experiments.

TABLE 5-6
Summary of solid vapor diffusion experiments

| Experiment ID | Solvent | Volume | Solid Form |
|---|---|---|---|
| 6022369-06-A1 | Acetone | 3 mL | Type X |
| 6022369-06-A2 | DMF | 3 mL | Type X |
| 6022369-06-A3 | DMSO | 3 mL | Type X |
| 6022369-06-A4 | EtOAc | 3 mL | Type X |
| 6022369-06-A5 | MeTHF | 3 mL | Type X |
| 6022369-06-A6 | MIBK | 3 mL | Type X |
| 6022369-06-A7 | NMP | 3 mL | Type X |
| 6022369-06-A8 | DMF/Ethyl | 3 mL | Type X |
| 6022369-06-A9 | DMSO/Ethyl | 3 mL | Type X |
| 6022369-06-A10 | Acetone/DMF 2:3 | 3 mL | Type X |

### 5.5 Liquid Vapor Diffusion

A total of 8 liquid vapor diffusion experiments were carried out. For each experiment, approximately 20 mg of starting material were weighed in a 4 mL glass vial and dissolved in correspondent solvent to obtain a clear solution. This vial was not sealed and was placed inside a 20-mL vial that was filled with approximately 3 mL of volatile anti solvent. The 20 mL vial was then sealed with a cap and kept at RT for 7 days, allowing the antisolvent vapor to interact with the solution inside of the 4 mL vial. After 7 days, the solids were taken out of the vial for 4 min XRPD analysis of the wet cakes. Vials in which no solids were observed, were set for slow evaporation for 5 days (the vial was covered with parafilm with 3-5 pinholes and left under RT for slow evaporation), and after this period, the resulting solids were taken out of the vial for 4 min XRPD analysis of the wet cakes.

Summary of experiments in Table 5-7 below shows the solvents and antisolvents used for each experiment, its corresponding volume, and indicates that Form X (i.e., Type X) and amorphous were the only solid form obtained in these experiments.

TABLE 5-7
Summary of liquid vapor diffusion experiments

| Experiment ID | Solvent | Volume | Anti-solvent | Volume | Solid Form |
|---|---|---|---|---|---|
| 6022369-07-A1 | DMF | 200 uL | H₂O | 3 mL | Type X |
| 6022369-07-A2 | DMF | 200 uL | Toluene | 3 mL | Clear solution** |
| 6022369-07-A3 | DMSO | 400 uL | H₂O | 3 mL | Amorphous |
| 6022369-07-A4 | DMSO | 400 uL | Toluene | 3 mL | Type X** |
| 6022369-07-A5 | Acetone | 200 uL | n-Heptane | 3 mL | Type X |
| 6022369-07-A6 | Acetone | 200 uL | Pentane | 3 mL | Type X |
| 6022369-07-A7 | EtOAc | 800 uL | n-Heptane | 3 mL | Type X |
| 6022369-07-A8 | EtOAc | 800 uL | Toluene | 3 mL | Type X* |

*Sample generated no solids, so it was set for evaporation for 5 days
**After one week the samples were moved to 5° C. for 3 days.

### 5.6 Slow Evaporation

A total of 10 slow evaporation experiments were carried out. For each experiment, approximately 20 mg of starting material were weighed in a 2 mL vial then dissolved in 0.2 mL of corresponding solvent, to obtain a clear solution. The vials were then covered by Parafilm® with 3 pinholes and subjected to evaporation at room temperature for 7 days. After this period, no solids were observed, so the samples were moved to 5° C. for 3 days to induce precipitation.

The resulting solids were taken out of the vial for 4 min XRPD analysis of the wet cakes. Summary of experiments in Table 5-8 below shows the solvents used for each experiment, its corresponding volume, and indicates that Form X (i.e., Type X) and Amorphous were obtained in these experiments.

TABLE 5-8
Summary of slow evaporation experiments

| Experiment ID | Solvent (v:v) | Volume | Solid Form |
|---|---|---|---|
| 6022369-08-A1 | NMP | 100 uL | Type X |
| 6022369-08-A2 | DMF/Ethyl | 100 uL | Type X |
| | Acetate 3:2 | | |
| 6022369-08-A3 | DMSO/Ethyl | 100 uL | Type X |
| | Acetate 3:2 | | |
| 6022369-08-A4 | Acetone | 100 uL | Type X |
| 6022369-08-A5 | DMF | 100 uL | Clear* |
| 6022369-08-A6 | DMSO | 100 uL | Amorphous |
| 6022369-08-A7 | EtOAc | 500 uL | Type X |
| 6022369-08-A8 | MeTHF | 300 uL | Type X |
| 6022369-08-A9 | MIBK | 500 uL | Type X |
| 6022369-08-A10 | NMP | 100 uL | Clear* |

*Because of the low volatility of DMF and NMP the samples were transferred to 5° C. to induce precipitation after 7 days of slow evaporation, but no crystallization occurred.

### 5.7 Ionic Liquid

A total of 10 ionic liquid experiments were carried out. For each experiment, approximately 20 mg of starting material were weighed in a 2 mL glass vial then dissolved in 0.1 mL of corresponding solvent to obtain a clear solution. About 5 mg of ionic liquid 1-Butyl-3-methyl imidazolium chloride were added into each glass vial. All the samples were subjected to evaporation at room temperature to induce precipitation for 7 days. No solids were observed after this period, so all samples were moved to 5° C. for 3 days.

The resulting solids were taken out of the vial for dry cake 4 min XRPD analysis. Summary of experiments in Table 5-9 below shows the solvents used for each experiment, its corresponding volume, and indicates that Form X (i.e., Type X) and Amorphous were obtained in these experiments.

TABLE 5-9
Summary of Ionic Liquid crystallization experiments

| Experiment ID | Solvent (v/v) | Volume | Solid Form |
|---|---|---|---|
| 6022369-09-A1 | Acetone | 200 uL | Type X |
| 6022369-09-A2 | DMF | 200 uL | Type X |
| 6022369-09-A3 | DMSO | 200 uL | Amorphous |
| 6022369-09-A4 | EtOAc | 800 uL | Type X |
| 6022369-09-A5 | MeTHF | 200 uL | Type X |
| 6022369-09-A6 | MIBK | 800 uL | Type X |
| 6022369-09-A7 | NMP | 200 uL | Clear solution |
| 6022369-09-A8 | Acetone/DMF 2:3 | 200 uL | Type X |
| 6022369-09-A9 | Acetone/DMSO 2:3 | 200 uL | Low crystalline* |
| 6022369-09-A10 | DMF/Ethyl | 200 uL | Type X |
| | Acetate 3:2 | | |

*XRPD only shows one or two reflection peaks but no amorphous halo was observed.
Based on the limited reflection, Type X assignment was not made.

### 5.8 Slow Cooling

A total of 10 slow cooling experiments were carried out. For each experiment, approximately 20 mg of starting material were weighed in a 2 mL glass vial then 0.1 mL of correspondent solvent were added to form saturated solutions. Those solutions were then cooled from 50° C. to 5° C. over a period of 7.5 hrs inside of an incubator. After this period, all solutions were clear (no solids). They were then set to slow evaporate at room temperature for 5 days. No solids were observed. They were moved to −14° C. for 3 days. After this period, the resulting solids were taken out of the vial for dry cake 4 min XRPD analysis.

Summary of experiments in Table 5-10 below shows the solvents used for each experiment, its corresponding volume, and indicates that only Form X (i.e., Type X) was obtained in these experiments.

TABLE 5-10
Summary of slow cooling experiments.

| Experiment ID | Solvent (v:v) | Volume | Solid Form |
|---|---|---|---|
| 6022369-10-A1 | DMF/EtOAc 3:2 | 200 uL | Clear solution* |
| 6022369-10-A2 | DMSO/EtOAc 3:2 | 200 uL | Clear solution* |
| 6022369-10-A3 | Acetone/DMF 2:3 | 200 uL | Clear solution* |
| 60223 69-1 0-A4 | Acetone/DMSO 2:3 | 200 uL | Type X |
| 6022369-10-A5 | DMSO | 100 uL | Clear solution* |
| 6022369-10-A6 | EtOAc | 100 uL | Type X |
| 6022369-10-A7 | MeTHF | 100 uL | Type X |
| 6022369-10-A8 | MIBK | 100 uL | Type X |
| 6022369-10-A9 | NMP | 100 uL | Clear solution* |
| 6022369-10-A10 | DMF | 100 uL | Clear solution* |

*No solids obtained after slow cooling.

The data showed that in all these conditions the only crystalline form of compound of Formula II is Form X. No new polymorph was observed during the screening. Therefore, it is concluded that Form X is the most stable form under the conditions tested in this study. The molecular structure was determined by single crystal X-ray diffraction.

### Example 6. Study of the Stability of Compound of Formula II Wet Cake

The wet cake drying study on compound of Formula II was carried out on a sample (98.67% purity of compound of Formula II) in an open dish held at 40° C. under vacuum. The sample was analyzed after one week (98.79% purity of compound of Formula II) and since the sample was stable, the study was extended another month. The solid can be held for at least a month under vacuum at 40° C. (98.74% purity of compound of Formula II).

### Example 7. Characterization of Compound of Formula III in the Amorphous Form

The amorphous sample of Compound of Formula III was obtained via rotary evaporation of methanol solution, and its XRPD was shown in FIG. 9. The DSC curve of amorphous sample is shown in FIG. 10. As shown in FIG. 10, no obvious glass transition signal was observed in the DSC of the amorphous sample.

### Example 8. Characterization of Compound of Formula III in the Crystalline Form

### 8.1 Anhydrate Form II of Compound of Formula III

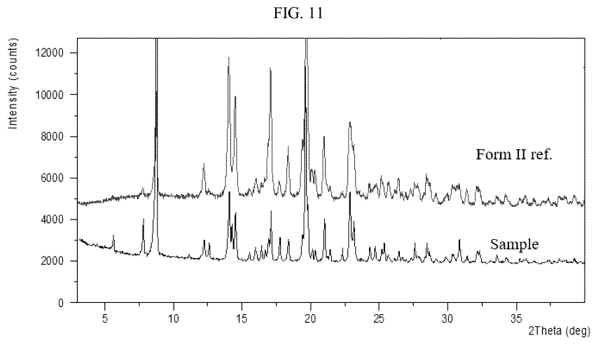

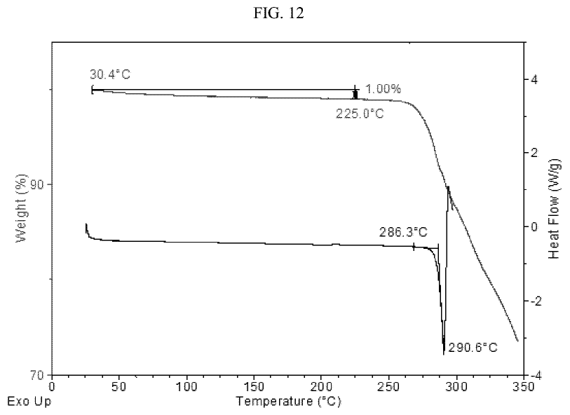

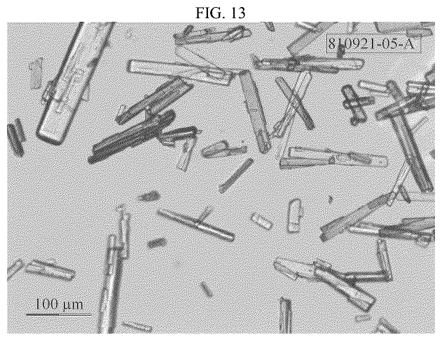

Anhydrate Form II of compound of Formula III was characterized by XRPD, TGA, DSC and HPLC. As shown in XRPD result (FIG. 11), the sample was crystalline and conformed to Form II. Its HPLC purity was determined to be 99.78 area %. As shown in FIG. 12, a weight loss of 1.0% before 225° C. in TGA and one sharp endothermic peak at 286.3° C. (onset temperature) in DSC were observed. The PLM image shows rods-like crystals (FIG. 13). Form II is the stable form of compound of Formula III.

### 8.2 Hydrate Form I of Compound of Formula III

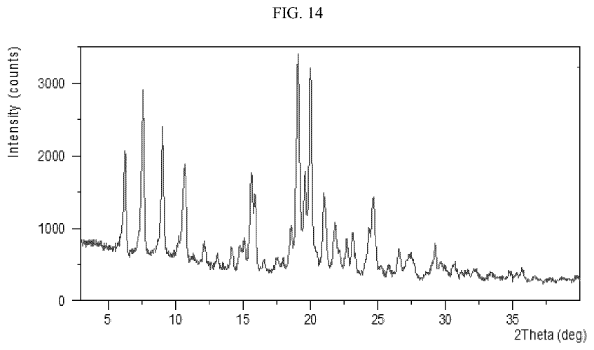

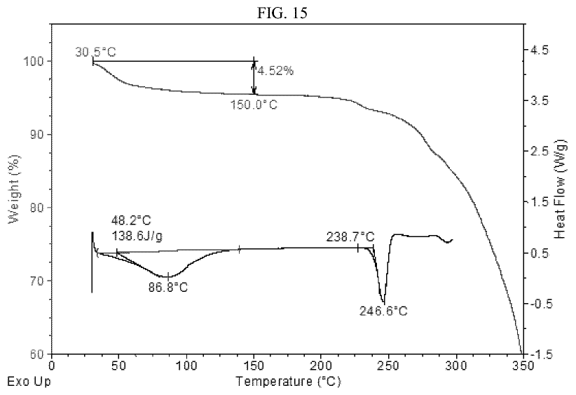

Form I of compound of Formula III was obtained via slurry of Form II in MeOH and then air dried for about 1 hr before characterization. Its HPLC purity was determined to be 99.83 area %. XRPD pattern is shown in FIG. 14, and TGA/DSC curves are displayed in FIG. 15. The results indicated that Form I was crystalline with a weight loss of 4.5% before 150° C. in TGA and two endothermic peaks at 48.2 and 238.7° C. in DSC (onset temperature).

### REFERENCES

Abetz-Webb L, Kennedy C, Hepburn B, Gauthier M, Johnson N, Medendorp S, et al. The burden of pruritus on patients with Alagille syndrome: results from a qualitative study with pediatric patients and their caregivers. Hepatology. 2014; 60:526A-527A.
Alissa F T, Jaffe R, Shneider B L. Update on progressive familial intrahepatic cholestasis. Journal of pediatric gastroenterology and nutrition. 2008; 46(3):241-52.
Arnell, H., Papadogiannakis, N., Zemack, H., Knisely, A. S., Nemeth, A. and Fischler, B. Follow-up in children with progressive familial intrahepatic cholestasis after partial external biliary diversion. J Pediatr Gastroenterol Nutr. 2010; 51:494-9.
Coates A, Nostrant T, Wilson J, Dobbins W, Agha F. Gastric xanthomatosis and cholestasis. A causal relationship. Dig Dis Sci. 1986; 31(9):925-928.
Danks D M, Campbell P E, Jack I, Rogers J, Smith A L. Studies of the aetiology of neonatal hepatitis and biliary atresia. Archives of disease in childhood. 1977; 52(5):360-7.
Davit-Spraul, A., Gonzales, E., Baussan, C. and Jacquemin, E. Progressive familial intrahepatic cholestasis. Orphanet J Rare Dis. 2009; 4:1.
Davit-Spraul A, Fabre M, Branchereau S, Baussan C, Gonzales E, Stieger B, et al. ATP8B1 and ABCB11 analysis in 62 children with normal gamma-glutamyl transferase progressive familial intrahepatic cholestasis (PFIC): phenotypic differences between PFIC1 and PFIC2 and natural history. Hepatology (Baltimore, Md). 2010; 51(5):1645-55.
Elisofon S A, Emerick K M, Sinacore J M, Alonso E M. Health status of patients with Alagille syndrome. Journal of pediatric gastroenterology and nutrition. 2010; 51(6):759-65.
Emerick K, Rand E, Goldmuntz E, Krantz I, Spinner N, Piccoli D. Features of Alagille syndrome in 92 patients: frequency and relation to prognosis. Hepatology. 1999; 29(3):822-829.
Emerick K M, Whitington P F. Partial external biliary diversion for intractable pruritus and xanthomas in Alagille syndrome. Hepatology (Baltimore, Md). 2002; 35(6):1501-6.
Emerick K M, Elias M S, Melin-Aldana H, Strautnieks S, Thompson R J, Bull L N, et al. Bile composition in Alagille Syndrome and PFIC patients having Partial External Biliary Diversion. BMC gastroenterology. 2008; 8:47.
Englert C, Grabhorn E, Richter A, Rogiers X, Burdelski M, Ganschow R. Liver transplantation in children with progressive familial intrahepatic cholestasis. Transplantation. 2007; 84(10):1361-3.
Halaweish, I. and Chwals, W. J. Long-term outcome after partial external biliary diversion for progressive familial intrahepatic cholestasis. J Pediatr Surg. 2010; 45:934-7.
Jericho, H. Bile Acid Pool Dynamics in Progressive Familial intrahepatic Cholestasis With Partial External Bile Diversion. Journal of Pediatric Gastroenterology and Nutrition. 2015; 60:368-374.
Kamath B M, Abetz-Webb L, Kennedy C, et al. Development of a novel tool to assess the impact of itching in pediatric cholestasis. Patient. 2018a;11:69-82.
Kamath B M, Baker A, Houwen R, Todorova L, Kerkar N. Systematic review: the epidemiology, natural history, and burden of Alagille syndrome. J Pediatr Gastroenterol Nutr. 2018b; 67(2):148-156.
Modi B, Suh M, Jonas M, Lillehei C, Kim H. Ileal exclusion for refractory symptomatic cholestasis in Alagille syndrome. J Pediatr Surg. 2007; 42(5):800-805.
Perez M, Briz O. Bile-acid-induced cell injury and protection. World J Gastroenterol. 2009; 15(14):1677-1689.
Sambrotta, M. Mutations in TJP2 cause progressive cholestatic liver disease. Nature Genetics. 2014; 46, 326-328,0023-0024.
Schukfeh, N., Metzelder, M. L., Petersen, C., Reismann, M., Pfister, E. D., Ure, B. M. and Kuebler, J. F. Normalization of serum bile acids after partial external biliary diversion indicates an excellent long-term outcome in children with progressive familial intrahepatic cholestasis. J Pediatr Surg. 2012; 47, 501-5.
Sira A, Sira M. Progressive familial intrahepatic cholestasis. In: Abdeldayem H, editor. Hepatic Surgery. InTech, DOI: 10.5772/51769 [cited 2017 Feb. 2]. Available from:
   http://www intechopen.com/books/hepatic-surgery/progressive-familial-intrahepatic-cholestasis. 2013.
Yang, H., Porte, R. J., Verkade, H. J., De Langen, Z. J. and Hulscher, J. B. 2009. Partial external biliary diversion in children with progressive familial intrahepatic cholestasis and Alagille disease. J Pediatr Gastroenterol Nutr, 49, 216-21.
Zhou S, Hertel P M, Finegold M J, Wang L, Kerkar N, Wang J, et al. Hepatocellular carcinoma associated with tight junction protein 2 deficiency. Hepatology (Baltimore, Md). 2015; 62(6):1914-6.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings. Accordingly, the present description is intended to embrace all such alternatives, modifications, and variances which fall within the scope of the appended claims.

All patents, applications, publications, test methods, literature, and other materials cited herein are hereby incorporated by reference in their entirety as if physically present in this specification.

1-260. (Canceled)

261. A crystalline form of (4R,5R)-3,3-dibutyl-7-(dimethylamino)-4-hydroxy-5-(4-methoxyphenyl)-2,3,4,5-tetrahydrobenzo[b]thiepine 1,1-dioxide (Formula I) 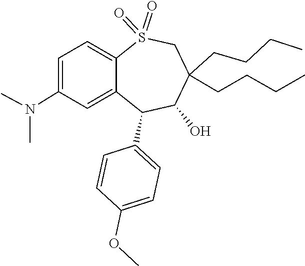 [SMILES: CCCCC1(CCCC)CS(=O)(=O)c2ccc(N(C)C)cc2[C@@H](c2ccc(OC)cc2)[C@H]1O]
or a pharmaceutically acceptable salt, hydrate, or solvate thereof, which is Form A, characterized by an X-ray powder diffraction diffractogram (XRPD) having a signal of at least three peaks in 2θ values chosen from 4.7±0.2, 6.6±0.2, 9.3±0.2, 10.4±0.2, 13.3±0.2, 16.8±0.2, 19.3±0.2, and 23.4±0.2.

262. The crystalline Form A according to claim 261, wherein the crystalline form is characterized by a melting point at about 133° C. to about 135° C., wherein the crystalline form is characterized by a DSC thermogram with an endotherm having an onset at about 130.8° C., a peak at about 134.7° C., and an exotherm at about 145° C., wherein the crystalline form has no weight loss between about 25° C. to about 225° C. as measured by thermogravimetric analysis (TGA), and/or wherein the crystalline form comprises rod-shaped crystals as observed by polarized light microscopy.

263. The crystalline Form A according to claim 261, wherein at least about 95% of the crystalline form is the R,R stereoisomer.

264. The crystalline Form A according to claim 261, wherein the purity of the compound of Formula I is at least about 95%.

265. A process of preparing the crystalline Form A according to claim 261, wherein the process comprises (a) mixing the compound of Formula I with a solvent to obtain a clear solution of the mixture, and (b) contacting the solution with a vapor of a volatile antisolvent.

266. The process of claim 265, wherein the solvent in step (a) comprises one or more selected from the group consisting of acetone, acetonitrile (ACN), n-butyl acetate, ethanol, cyclohexane, dichloromethane (DCM), dimethylformamide (DMF), dimethylsulfoxide (DMSO), ethyl acetate (EtOAc), ethanol (EtOH), water, n-heptane, isopropyl alcohol (IPA), isopropyl acetate (IPAc), methanol (MeOH), methyltetrahydrofuran (MeTHF), methyl isobutyl ketone (MIBK), methyl tert-butyl ether (MTBE), N-methyl-2-pyrrolidone (NMP), pentane, and toluene, and/or wherein the antisolvent in step (b) comprises one or more selected from the group consisting of cyclohexane, water, n-heptane, IPA, MTBE, and pentane.

267. A crystalline form of (4R,5R)-3,3-dibutyl-7-(dimethylamino)-4-hydroxy-5-(4-hydroxyphenyl)-2,3,4,5-tetrahydrobenzo[b]thiepine 1,1-dioxide (Formula II) 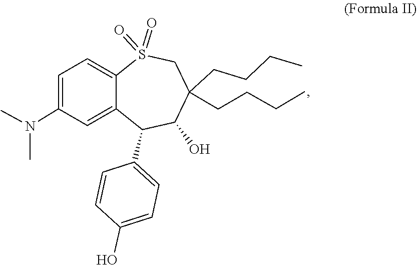 [SMILES: CCCCC1(CCCC)CS(=O)(=O)c2ccc(N(C)C)cc2[C@@H](c2ccc(O)cc2)[C@H]1O]
or a pharmaceutically acceptable salt, hydrate, or solvate thereof, which is Form X, characterized by an X-ray powder diffractogram (XRPD) having a signal of at least nine peaks in 2θ values chosen from 5.0±0.2, 8.7±0.2, 10.0±0.2, 12.5±0.2, 13.3±0.2, 14.9±0.2, 15.8±0.2, 17.0±0.2, 17.8±0.2, 19.3±0.2, 19.9±0.2, 21.7±0.2, 22.6±0.2, 23.2±0.2, 24.5±0.2, 24.9±0.2, 25.8±0.2, 26.4±0.2, 28.0±0.2, and 29.4±0.2.

268. The crystalline Form X according to claim 267, wherein the crystalline form is characterized by a melting point at about 216.7° C. to about 217.7° C., or wherein the crystalline form is characterized by a DSC thermogram with an endotherm having an onset at about 216.1° C., a peak at about 217.2° C., and an exotherm at about 218.3° C., wherein the crystalline form has less than about 0.1% weight loss between about 95.1° C. to about 200.0° C. as measured by thermogravimetric analysis (TGA), and/or wherein the crystalline form comprises rod-shaped crystals as observed by polarized light microscopy.

269. The crystalline Form X according to claim 267, wherein the crystalline form comprises rod-shaped crystals as observed by polarized light microscopy.

270. The crystalline Form X according to claim 267, wherein at least about 95% of the crystalline form is the R,R stereoisomer.

271. The crystalline Form X according to claim 267, wherein the purity of the compound of Formula I is at least about 95%.

272. A process of preparing the crystalline Form X according to claim 267, wherein the process comprises (a) mixing the compound of Formula II with a solvent to obtain a clear solution of the mixture, and (b) contacting the solution with a vapor of a volatile antisolvent.

273. The process of claim 272, wherein the solvent in step (a) comprises one or more selected from the group consisting of acetone, acetonitrile (CAN), n-butyl acetate, ethanol, cyclohexane, dichloromethane (DCM), dimethylformamide (DMF), dimethylsulfoxide (DMSO), ethyl acetate (EtOAc), ethanol (EtOH), water, n-heptane, isopropyl alcohol (IPA), isopropyl acetate (IPAc), methanol (MeOH), methyltetrahydrofuran (MeTHF), methyl isobutyl ketone (MIBK), methyl tert-butyl ether (MTBE), N-methyl-2-pyrrolidone (NMP), pentane, and toluene, and/or wherein the antisolvent in step (b) comprises one or more selected from the group consisting of toluene, water, n-heptane, and pentane.

274. A process of making a highly pure compound 1-(4-((4-((4R,5R)-3,3-dibutyl-7-(dimethylamino)-4-hydroxy-1,1-dioxido-2,3,4,5-tetrahydrobenzo[b]thiepin-5-yl)phenoxy)methyl)benzyl)-1,4-diazabicyclo[2.2.2] octan-1-ium chloride (Formula III) 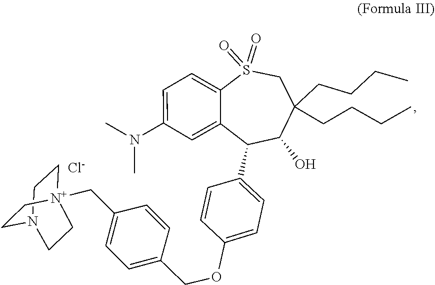 [SMILES: CCCCC1(CCCC)CS(=O)(=O)c2ccc(N(C)C)cc2[C@@H](c2ccc(OCc3ccc(C[N+]45CCN(CC4)CC5)cc3)cc2)[C@H]1O ; [Cl-]]
said process comprising the step of converting a crystalline form of a compound of Formula I, or a pharmaceutically acceptable salt, hydrate, or solvate thereof, to a crystalline form of a compound of Formula II, or a pharmaceutically acceptable salt thereof, wherein the crystalline form of the compound of Formula I, or a pharmaceutically acceptable salt, hydrate, or solvate thereof, is characterized by an X-ray powder diffraction diffractogram (XRPD) having a signal of at least three peaks in 2θ values chosen from 4.7±0.2, 6.6±0.2, 9.3±0.2, 10.4±0.2, 13.3±0.2, 16.8±0.2, 19.3±0.2, and 23.4±0.2, and wherein the crystalline form of the compound of Formula II, or a pharmaceutically acceptable salt, hydrate, or solvate thereof, is characterized by an X-ray powder diffractogram (XRPD) having a signal of at least nine peaks in 2θ values chosen from 5.0±0.2, 8.7±0.2, 10.0±0.2, 12.5±0.2, 13.3±0.2, 14.9±0.2, 15.8±0.2, 17.0±0.2, 17.8±0.2, 19.3±0.2, 19.9±0.2, 21.7±0.2, 22.6±0.2, 23.2±0.2, 24.5±0.2, 24.9±0.2, 25.8±0.2, 26.4±0.2, 28.0±0.2, and 29.4±0.2, and wherein the purity of the compound of Formula III is at least about 90%.

275. The process of making the highly pure compound of Formula III according to claim 274, wherein the compound of Formula III is in a crystalline form.

276. The process of making the highly pure compound of Formula III according to claim 274, wherein the compound of Formula III is in crystalline Form II.

277. A highly pure compound of Formula III, wherein the compound is prepared by a process comprising the step of converting a crystalline form of a compound of Formula I, or a pharmaceutically acceptable salt, hydrate, or solvate thereof, to a crystalline form of a compound of Formula II, or a pharmaceutically acceptable salt thereof, wherein the crystalline form of the compound of Formula I, or a pharmaceutically acceptable salt, hydrate, or solvate thereof, is characterized by an X-ray powder diffraction diffractogram (XRPD) having a signal of at least three peaks in 2θ values chosen from 4.7±0.2, 6.6±0.2, 9.3±0.2, 10.4±0.2, 13.3±0.2, 16.8±0.2, 19.3±0.2, and 23.4±0.2, and wherein the crystalline form of the compound of Formula II, or a pharmaceutically acceptable salt, hydrate, or solvate thereof, is characterized by an X-ray powder diffractogram (XRPD) having a signal of at least nine peaks in 2θ values chosen from 5.0±0.2, 8.7±0.2, 10.0±0.2, 12.5±0.2, 13.3±0.2, 14.9±0.2, 15.8±0.2, 17.0±0.2, 17.8±0.2, 19.3±0.2, 19.9±0.2, 21.7±0.2, 22.6±0.2, 23.2±0.2, 24.5±0.2, 24.9±0.2, 25.8±0.2, 26.4±0.2, 28.0±0.2, and 29.4±0.2, and wherein the purity of the compound of Formula III is at least about 90%.

278. The compound of Formula III, wherein the compound is in crystalline Form II.

279. A method of treating cholestatic liver disease or condition or treating pruritus in a subject in need thereof, comprising administering a therapeutically effective amount of the highly pure compound of Formula III according to claim 277.

280. The method of claim 279, wherein the cholestatic liver disease or condition is selected from the group consisting of obstructive cholestasis, non-obstructive cholestasis, extrahepatic cholestasis, intrahepatic cholestasis, primary intrahepatic cholestasis, secondary intrahepatic cholestasis, progressive familial intrahepatic cholestasis (PFIC), PFIC type 1, PFIC type 2, PFIC type 3, benign recurrent intrahepatic cholestasis (BRIC), BRIC type 1, BRIC type 2, BRIC type 3, total parenteral nutrition associated cholestasis, paraneoplastic cholestasis, Stauffer syndrome, intrahepatic cholestasis of pregnancy, contraceptive-associated cholestasis, drug-associated cholestasis, infection-associated cholestasis, Dubin-Johnson Syndrome, primary biliary cirrhosis (PBC), primary sclerosing cholangitis (PSC), gallstone disease, Alagille syndrome, Dubin-Johnson Syndrome, biliary atresia, post-Kasai biliary atresia, post-liver transplantation biliary atresia, post-liver transplantation cholestasis, post-liver transplantation associated liver disease, intestinal failure associated liver disease, bile acid mediated liver injury, MRP2 deficiency syndrome, and neonatal sclerosing cholangitis, or wherein the pruritus is associated with Alagille syndrome (ALGS).
